SEQUENCE LISTING (10 sequences)

SEQ ID NO: 1 - protein, 447 aa, Artificial Sequence
  Synthetic peptide
QLQLQESGPGLVKPSETLSLTCTVSGDSISKNSYYWGWIRQPPGKGLEWIGSMYYSGSTYYNSSLKSRVTISVDTSKNQFSLKLSSVTAADTAVYYCARHDGGASIFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 212 aa, Artificial Sequence
  Synthetic peptide
SYELTQPPSVSVSPGQTASITCSGDKLGDMDACWYQQRPGQSPVVVIYQDSERPSGIPERFAGSNSGNTATLTISGTQAMDEADYYCQAWDSSTVVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 3 - protein, 452 aa, Artificial Sequence
  Synthetic peptide
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 4 - protein, 215 aa, Artificial Sequence
  Synthetic peptide
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 5 - protein, 448 aa, Artificial Sequence
  Synthetic peptide
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 214 aa, Artificial Sequence
  Synthetic peptide
SYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 7 - protein, 445 aa, Artificial Sequence
  Synthetic peptide
EVQLVQSGAEVKKPGESLKISCKGSGYSFTSYWISWVRQMPGKGLEWMGIIDPSDSDTRYSPSFQGQVTISADKSISTAYLQWSSLKASDTAMYYCARGDGSTDLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 215 aa, Artificial Sequence
  Synthetic peptide
EIVLTQSPGTLSLSPGERATLSCRASQSVSSSYLAWYQQKPGQAPRLLIYGASSRATGIPDRFSGSGSGTDFTLTISRLEPEDFAVYYCQQDYGFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 452 aa, Artificial Sequence
  Synthetic peptide
EVQLVESGGGLVQPGGSLKLSCAASGFTFNTYAMNWVRQASGKGLEWVGRIRSKYNAYATYYAASVKGRFTISRDDSKNTAYLQMNSLKTEDTAVYYCTRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 10 - protein, 215 aa, Artificial Sequence
  Synthetic peptide
QAVVTQEPSLTVSPGGTVTLTCRSSTGAVTTSNYANWVQQKPGQAPRGLIGGTNKRAPGTPARFSGSLLGGKAALTLSGAQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2021/0363252 A1
(43) Pub. Date: Nov. 25, 2021

(54) COMPOSITIONS COMPRISING A T CELL REDIRECTION THERAPEUTIC AND A VLA-4 ADHESION PATHWAY INHIBITOR

(71) Applicant: Janssen Biotech, Inc., Horsham, PA (US)

(72) Inventors: Priyanka Nair-Gupta, Spring House, PA (US); François Gaudet, Princeton, NJ (US)

(21) Appl. No.: 17/322,973

(22) Filed: May 18, 2021

Related U.S. Application Data
(60) Provisional application No. 63/026,885, filed on May 19, 2020.

(51) Int. Cl. C07K 16/28; A61P 35/00; A61K 45/06; A61K 39/395

(52) U.S. Cl. CPC C07K 16/2809; C07K 16/2878; C07K 16/2842; A61P 35/00; A61K 2039/507; A61K 39/3955; C07K 16/2866; C07K 2317/31; A61K 45/06

(57) ABSTRACT

Disclosed herein is a pharmaceutical composition comprising a T cell redirect therapeutic and a VLA-4 adhesion pathway inhibitor, and uses thereof for killing cancer cells.

### CROSS REFERENCE TO RELATED APPLICATIONS

This application claims the benefit of U.S. Provisional Application 63/026,885, filed on May 19, 2020, which is incorporated by reference herein in its entirety.

### FIELD OF THE INVENTION

This disclosure relates to compositions and killing cancer cells utilizing T cell redirection therapeutics.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created on Apr. 30, 2021, is named JBI6312USNP1_SL.txt and is 29 KB in size.

### BACKGROUND OF THE INVENTION

Despite several treatment options, there is currently no cure for acute myeloid leukemia (AML) and multiple myeloma (MM). Even after achieving high rates (50%-80%) of complete hematologic remission (CR), defined as the presence of ≤5% of leukemic blasts (AML) or plasma cells (MM) in the bone marrow (BM) (1, 2), the majority of patients with AML or MM relapse (3-5). Relapse has been linked to minimal residual disease (MRD) whereby small numbers of cancer stem cells (CSC), or other malignant progenitor cells, fail to be cleared and persist even after therapy (6). Preventing relapses and finding cures for AML and MM requires finding better strategies to eliminate MRD.

Like hematopoietic stem cells (HSC), CSC in AML and MM reside and preferentially persist in the BM niche (7, 8). The BM niche provides a specialized microenvironment via secretion of soluble growth factors and cell-cell interactions that are protective to the CSC (9). Moreover, the BM niche is immune-suppressive and is appreciated to be a site of immune privilege at steady state to allow for normal hematopoiesis and immune cell generation (10). These aspects of the BM niche have provided resistance against and minimized the efficacy of several anti-cancer drugs including chemotherapy, targeted small molecule inhibitors, and antibody based therapies (11-14).

The ability of T cells to specifically lyse tumor cells and secrete cytokines to recruit and support immunity against cancer makes them an attractive option for therapy. Several approaches have capitalized on this strategy such as bispecific T-cell engagers (BiTEs, small bispecific biologics), chimeric antigen receptors (CARs) and bispecific antibodies, among others (15). BiTEs and antibody-mediated redirection cross-link T cells to tumor cells by engaging a specific epitope on tumor cells and CD3 on T cells, leading to T cell activation, and secretion of perforins and granzymes that ultimately kill the tumor cells. These CD3 redirection therapies have been validated as an effective anti-cancer strategy in the clinic with the approval of CD19×CD3 BiTE (blinatumomab) for acute lymphoblastic lymphoma (ALL) (16). However, the immunosuppressive and protective nature of the BM niche potentially poses a significant hurdle to T cell redirection therapies.

For example, as shown herein, using bispecific antibodies targeting specific tumor antigens (CD123 and BCMA) and CD3, it was observed that co-culture of AML or MM cell lines with BM stromal cells significantly protected cancer cells from bispecific-T cell-mediated lysis in vitro. Similar results were observed in vivo when presence of human BM stromal cells in a humanized xenograft AML model attenuated tumor growth inhibition (TGI) observed with bispecific antibody treatment. Impaired CD3 redirection cytotoxicity was correlated with reduced T cell effector responses, thereby providing a mechanism to explain loss of activity of the bispecific antibody.

### BRIEF SUMMARY OF THE INVENTION

Provided herein is a pharmaceutical composition comprising a T cell redirection therapeutic and a VLA-4 adhesion pathway inhibitor, wherein, the T cell redirection therapeutic comprises a first binding region having specificity against a T cell surface antigen and a second binding region having specificity against a tumor associated antigen (TAA).

In one embodiment of the pharmaceutical composition, the composition further comprises a pharmaceutically acceptable carrier.

In a further embodiment of the pharmaceutical composition, the T cell redirection therapeutic is an antibody or antigen-binding fragment thereof.

In a yet further embodiment of the pharmaceutical composition, the T cell surface antigen is selected from the group consisting of CD3, CD2, CD4, CD5, CD6, CD8, CD28, CD40L, CD44, CD137, KI2L4, NKG2E, NKG2D, NKG2F, BTNL3, CD186, BTNL8, PD-1, CD195, and NKG2C.

In a yet further embodiment of the pharmaceutical composition, the T cell surface antigen is CD3.

In a yet further embodiment of the pharmaceutical composition, the TAA is selected from the group consisting of BCMA, CD123, GPRC5D, CD33, CD19, PSMA, TMEFF2, CD20, CD22, CD25, CD52, ROR1, HM1.24, CD38, and SLAMF7.

In a yet further embodiment of the pharmaceutical composition, the T cell surface antigen is a BCMAxCD3 bispecific antibody having a first antigen-binding site that immunospecifically binds BCMA and a second antigen-binding site that immunospecifically binds CD3.

In a yet further embodiment of the pharmaceutical composition, the BCMAxCD3 bispecific antibody comprises a first heavy chain (HC1), a first light chain (LC1), a second heavy chain (HC2), and a second light chain (LC2), and wherein the HC1 and the LC1 pair to form the first antigen-binding site and the HC2 and the LC2 pair to form the second antigen-binding site.

In a yet further embodiment of the pharmaceutical composition, the HC1 comprises the amino acid sequence of SEQ ID NO: 1, the LC1 comprises the amino acid sequence of SEQ ID NO: 2, the HC2 comprises the amino acid sequence of SEQ ID NO: 3, and the LC2 comprises the amino acid sequence of SEQ ID NO: 4.

In a yet further embodiment of the pharmaceutical composition, the HC1 comprises the amino acid sequence of SEQ ID NO: 5, the LC1 comprises the amino acid sequence of SEQ ID NO: 6, the HC2 comprises the amino acid sequence of SEQ ID NO: 3, and the LC2 comprises the amino acid sequence of SEQ ID NO: 4.

In a yet further embodiment of the pharmaceutical composition, the T cell surface antigen is a CD123×CD3 bispecific antibody having a first antigen-binding site that immunospecifically binds CD123 and a second antigen-binding site that immunospecifically binds CD3.

In a yet further embodiment of the pharmaceutical composition, the CD123×CD3 bispecific antibody comprises a first heavy chain (HC1), a first light chain (LC1), a second heavy chain (HC2), and a second light chain (LC2), and wherein the HC1 and the LC1 pair to form the first antigen-binding site and the HC2 and the LC2 pair to form the second antigen-binding site.

In a yet further embodiment of the pharmaceutical composition, the HC1 comprises the amino acid sequence of SEQ ID NO: 7, the LC1 comprises the amino acid sequence of SEQ ID NO: 8, the HC2 comprises the amino acid sequence of SEQ ID NO: 9, and the LC2 comprises the amino acid sequence of SEQ ID NO: 10.

In a yet further embodiment of the pharmaceutical composition, the VLA-4 adhesion pathway inhibitor is an anti-VLA-4 antibody or antigen-binding fragment thereof.

In a yet further embodiment of the pharmaceutical composition, the anti-VLA-4 antibody or antigen-binding fragment thereof is selected from the group consisting of monoclonal antibodies, scFv, Fab, Fab′, F(ab′)2, and F(v) fragments, heavy chain monomers or dimers, light chain monomers or dimers, and dimers consisting of one heavy chain and one light chain.

In a yet further embodiment of the pharmaceutical composition, the VLA-4 adhesion pathway inhibitor is a VLA-4 antagonist.

In a yet further embodiment of the pharmaceutical composition, the VLA-4 adhesion pathway inhibitor is a VLA-4 antagonist selected from the group consisting of BIO1211, TCS2314, BIO5192, and TR14035.

Further provided herein is a method of killing cancer cells, comprising administering a therapeutically effective amount of the pharmaceutical composition provided above.

In a further embodiment of the method, the cancer is a hematological malignancy or a solid tumor.

In a yet further embodiment of the method, the T cell redirection therapeutic and the VLA-4 adhesion pathway inhibitor are administered simultaneously or sequentially.

In a yet further embodiment of the method, the VLA-4 adhesion pathway inhibitor is administered prior to the T cell redirection therapeutic.

In a yet further embodiment of the method, the VLA-4 adhesion pathway inhibitor is administered after administration of the T cell redirection therapeutic.

Yet further provided herein is a kit comprising the pharmaceutical composition provided above.

### BRIEF DESCRIPTION OF THE DRAWINGS

The foregoing summary, as well as the detailed description and embodiments of the present application, will be better understood when read in conjunction with the appended claims and drawings. It should be understood, however, that the invention is not limited to the precise recitations disclosed herein.

FIGS. 1A-1D show that the presence of stromal cells protects AML and MM cell lines from T cell redirected cytotoxicity. Human T cells (40,000 cells/well) were cultured with CFSE labelled AML KG1 (FIG. 1A, FIG. 1B) or MM H929 cell lines (FIG. 1C, FIG. 1D) 2:1 ratio in the presence or absence of stromal cells (HS-5, HS-27a, primary MSC or CD105⁺ endothelial cells; 20,000 cells per well). Varying concentrations of CD123×CD3 (FIG. 1A, FIG. 1B) or BCMA×CD3 (FIG. 1C, FIG. 1D) were added to cultures for 48 hours. The percentage of dead CFSE⁺ cells was quantitated by flow cytometry. Dose titration graphs (FIG. 1A, FIG. 1C) are shown with means±standard deviation (SD). Scatter plots (FIG. 1B, FIG. 1D) show data for the highest concentration of the bispecific antibody (median with range). Data are representative of three or more experiments. ** p<0.005, *** p<0.0005; **** p<0.0001; n.s., not statistically significant.

FIGS. 2A-2D show that stromal cells suppress T cell function, upregulate signaling pathways in tumor cells and protect tumor cells from T cell redirected cytotoxicity. FIG. 2A: Human T cells (40,000 cells/well) were cultured with CFSE labelled KG-1 cells at a 2:1 ratio in the presence or absence of stromal cells (HS-5, primary MSC or CD105⁺ endothelial cells; 20,000 cells per well). Analysis of activation, effector and checkpoint inhibition markers in CD8⁺ T cells was performed post addition of 33 nM of CD123×CD3 to cultures. Geometric mean fluorescence intensities were quantified by flow cytometry at 48 hours. FIG. 2B: Immunoblotting analysis of activation (phosphorylation) of PI3K and Akt as well as expression of Bcl-2 in KG-1 cells cultured either alone or in the presence of HS-5 stromal cell line for 48 hours. FIG. 2C: Similar to FIG. 2A but here all the cultures were treated with or without Bcl-2i and the percentage of dead CFSE⁺ cells was quantitated by flow cytometry. FIG. 2D: Similar to FIGS. 2A and 2C where activation status of T cells was assessed in tumor and T cell cultures in the absence or presence of stroma and with or without treatment with Bcl-2i. All data shown are representative of three or more experiments and are depicted as either mean with SD (dose titration curves) or median with range (scatter plots). * p<0.05, ** p<0.005, *** p<0.0005; **** p<0.0001; n.s., not statistically significant.

FIGS. 3A-3C show that stromal cells impact efficacy of CD3 redirection in vivo. MOLM-13 AML and MOLM-13 with HS-5 bone marrow stromal cells (5:1) were implanted sc in huPBMC injected female NSG mice on study day 0. Mice were treated with CD123×CD3 (8 μg/kg) starting on day 5 post tumor cell implant twice weekly for a total of 5 treatments.

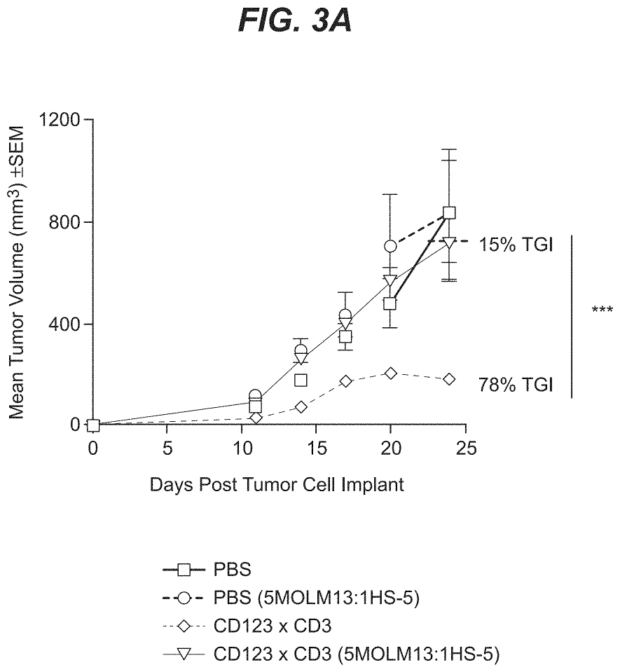

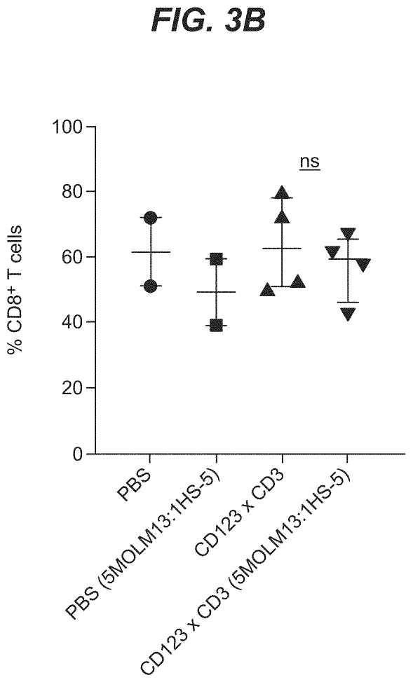

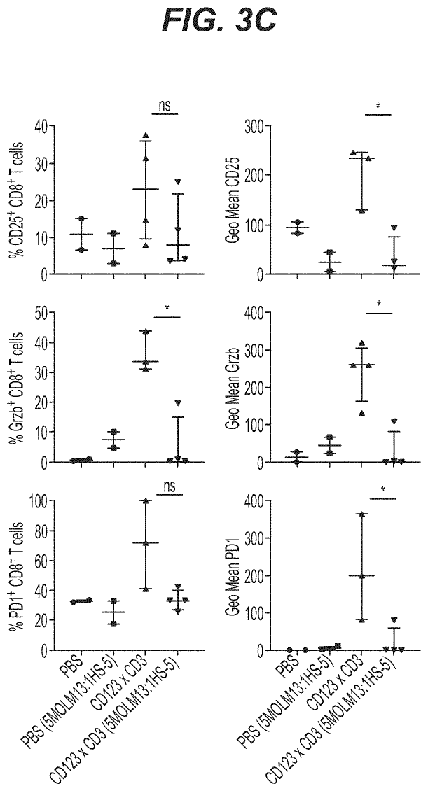

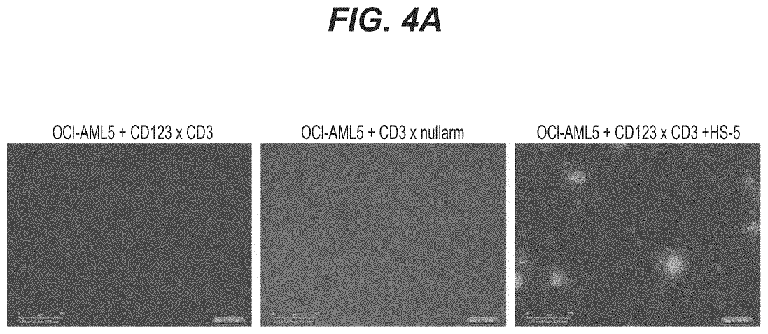

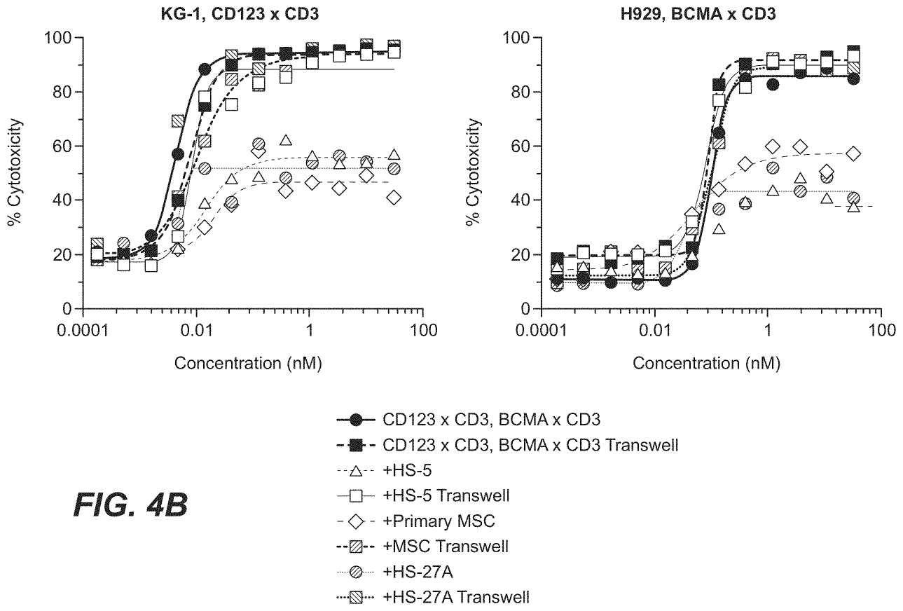

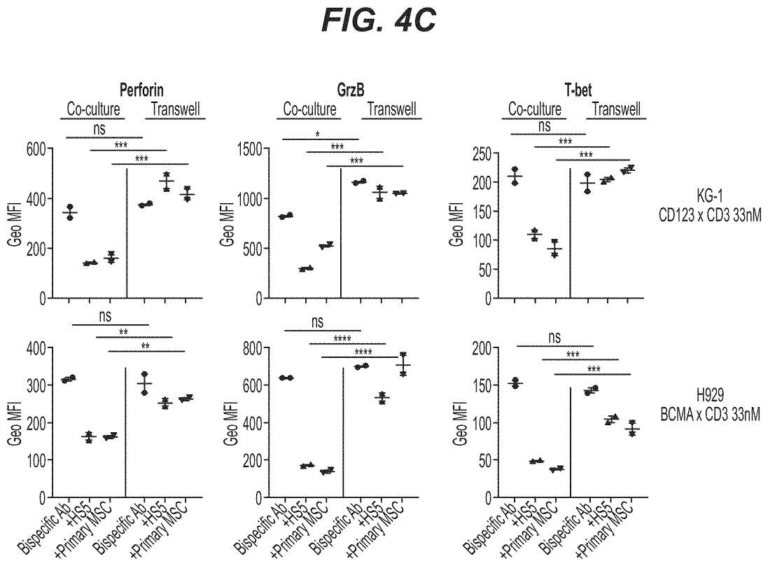

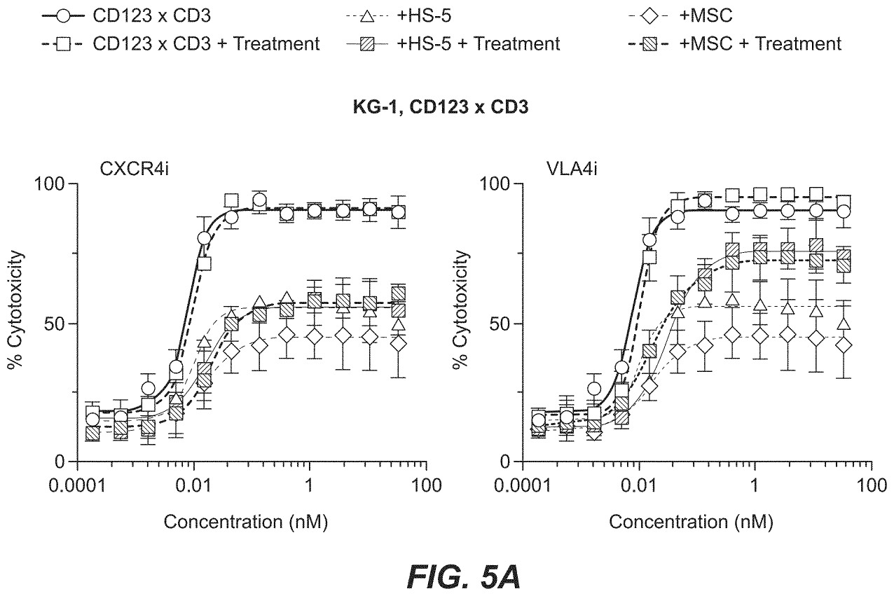

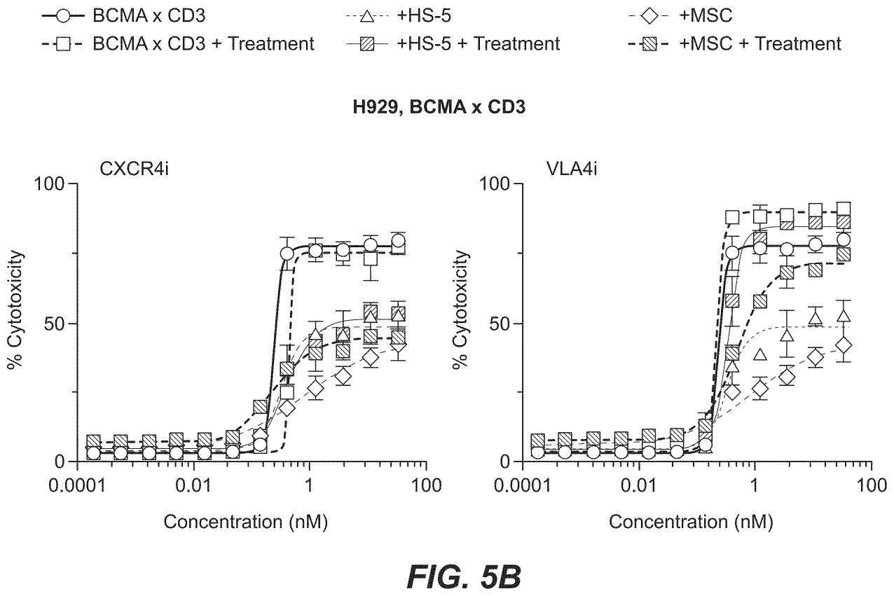

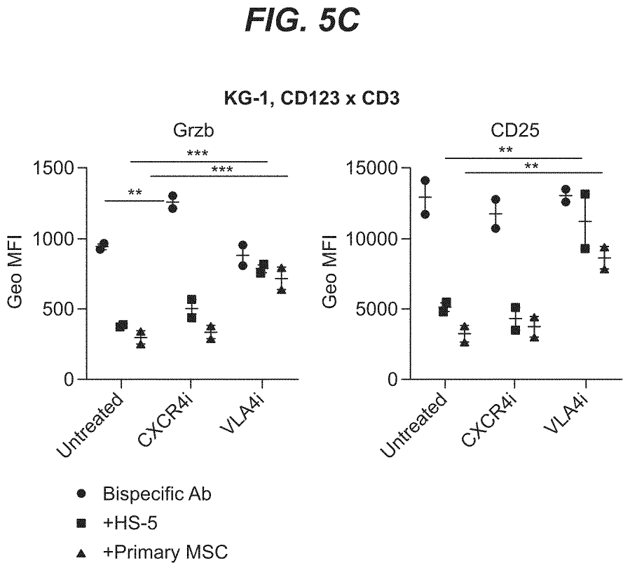

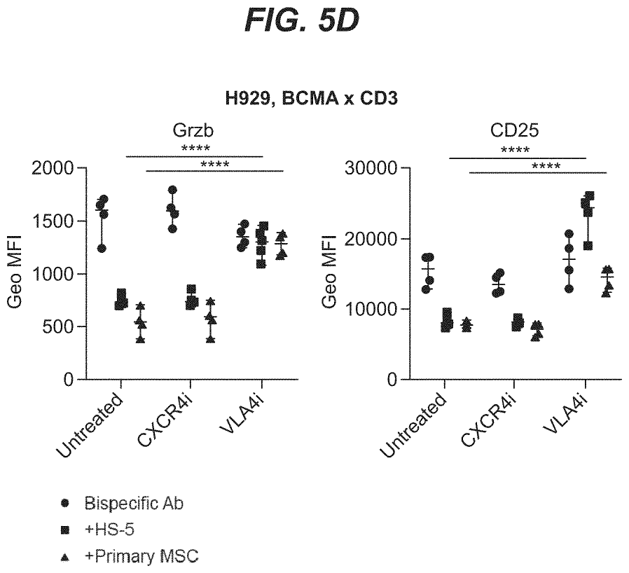

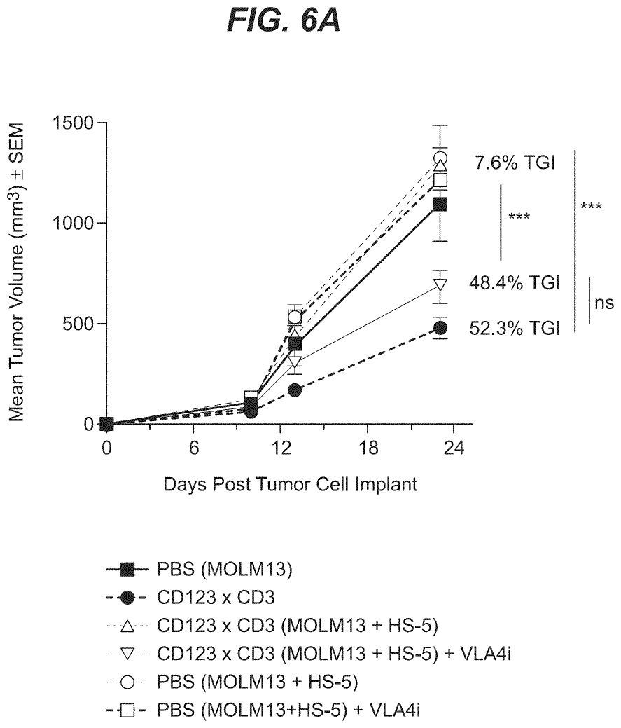

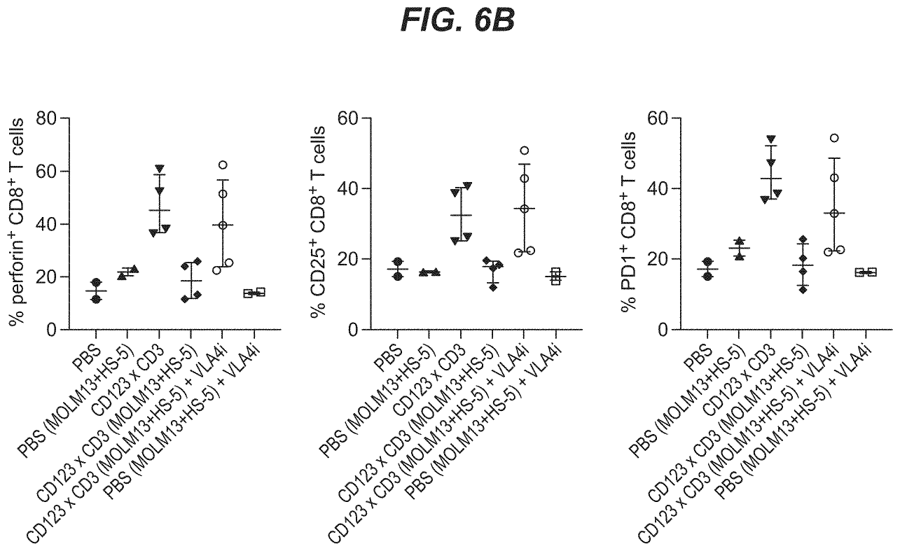

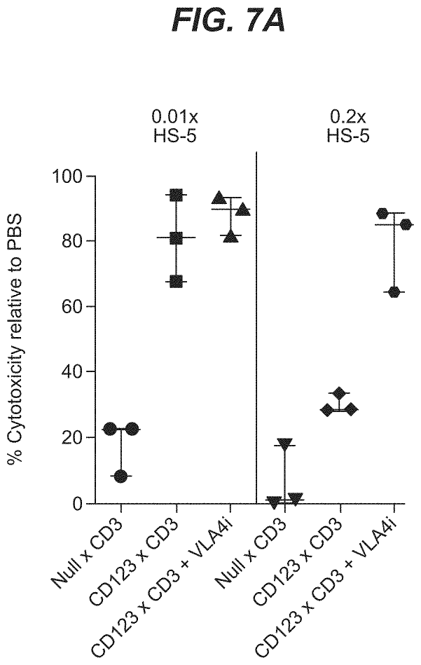

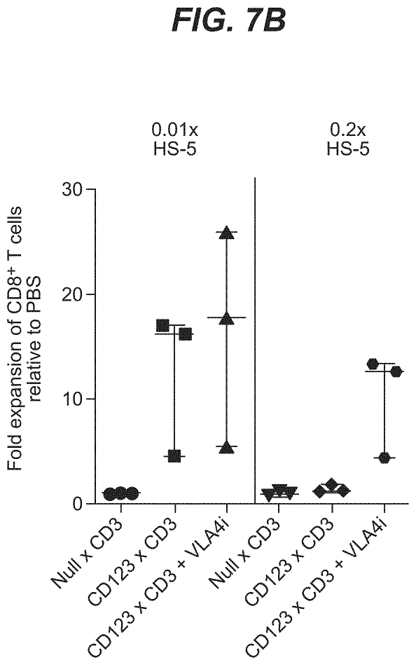

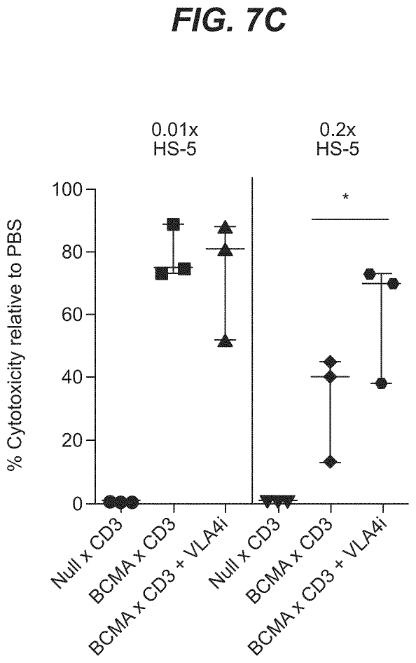

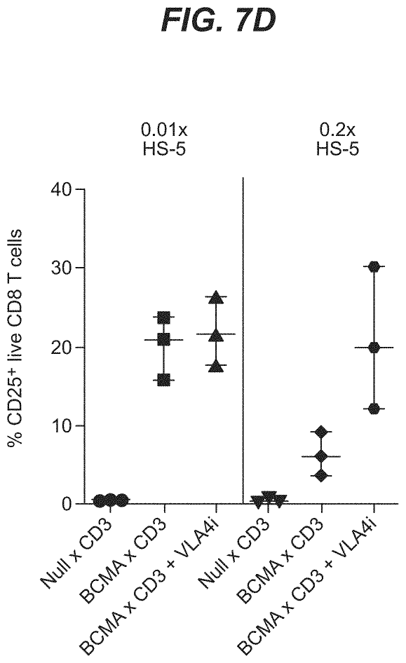

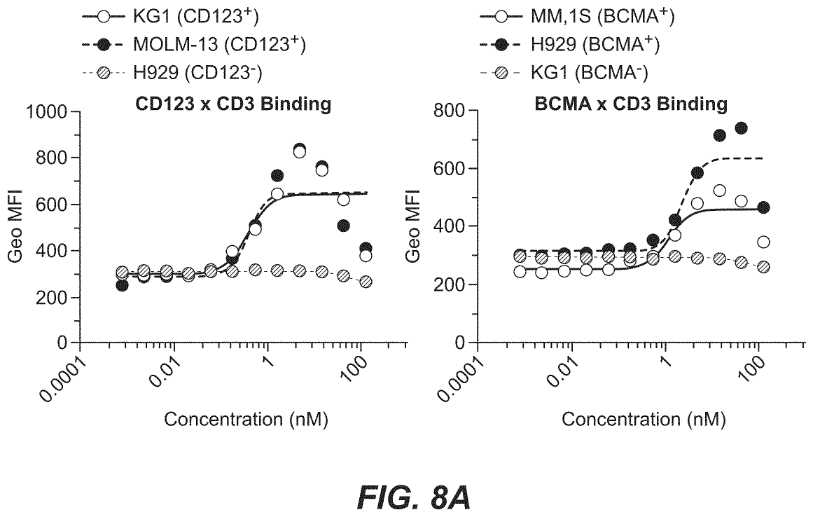

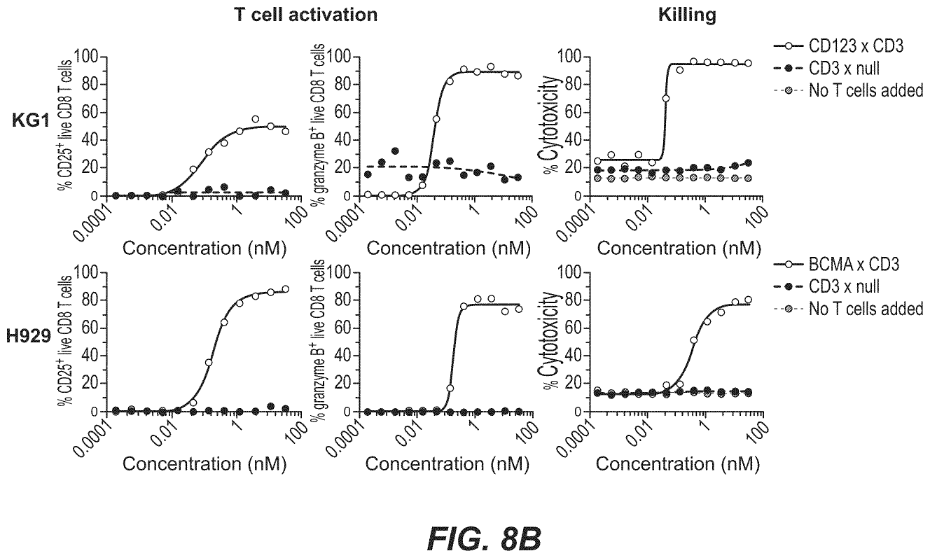

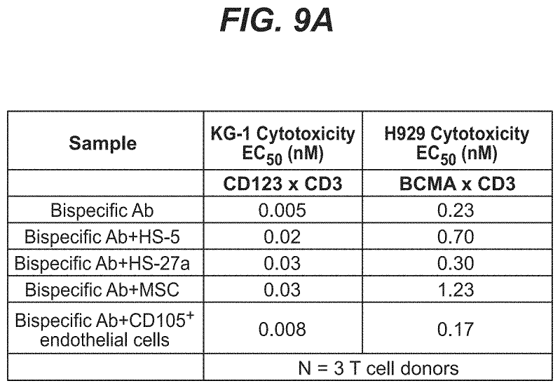

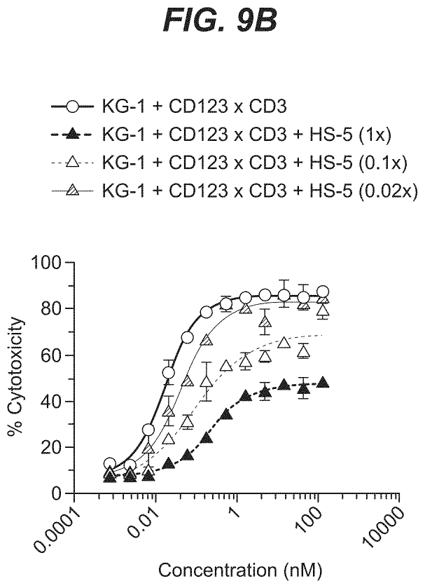

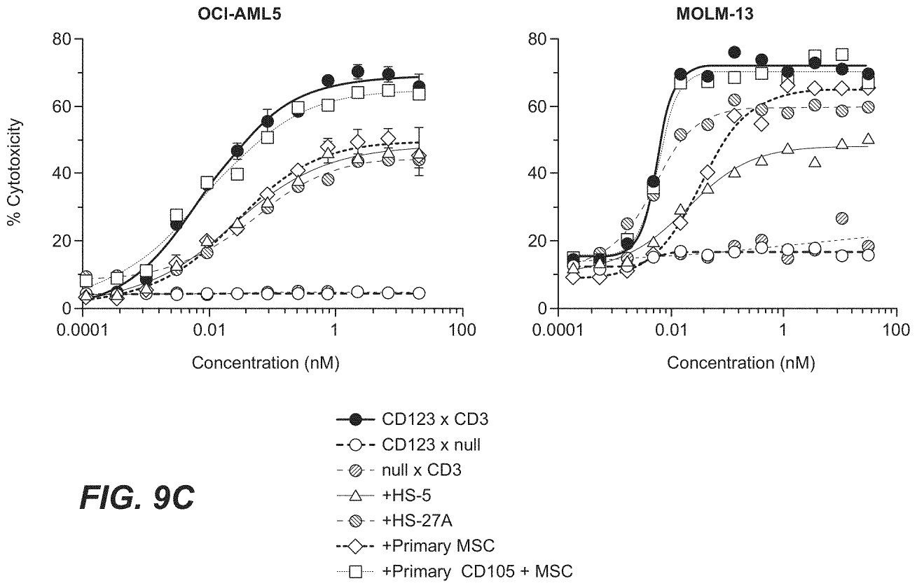

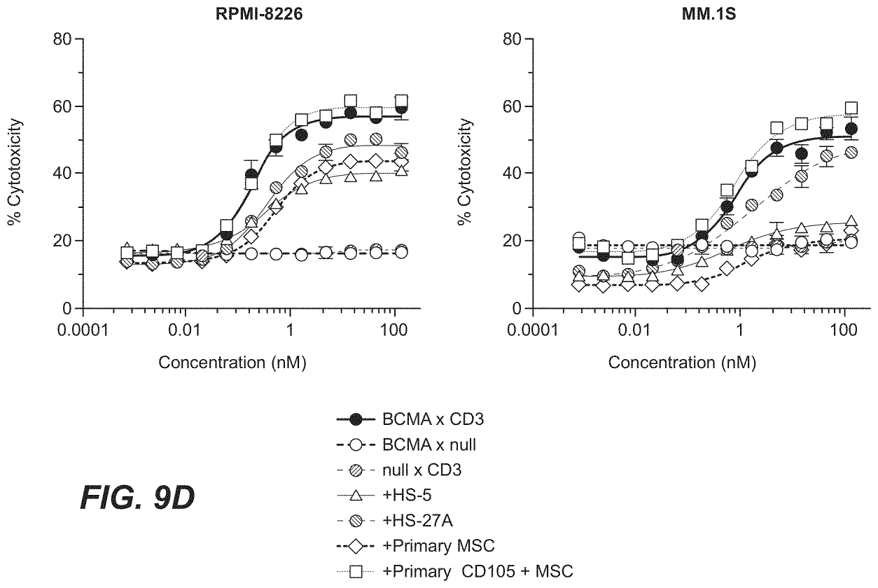

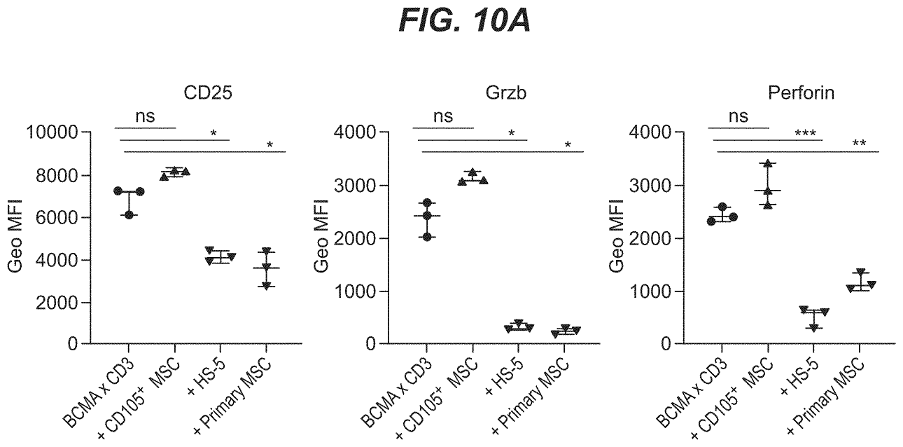

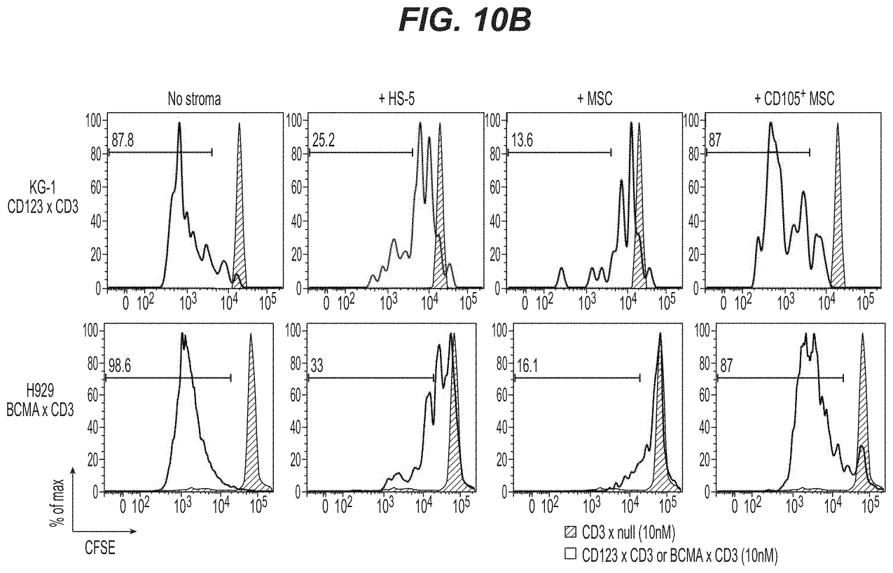

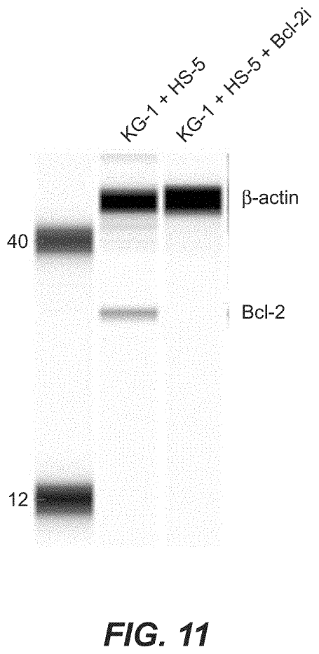

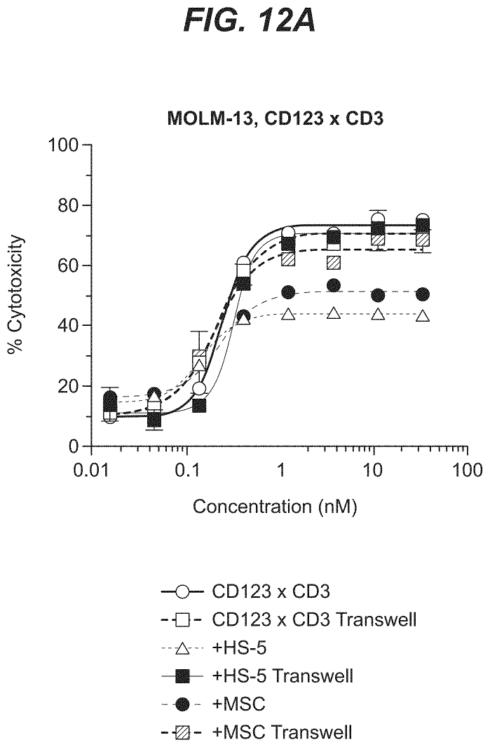

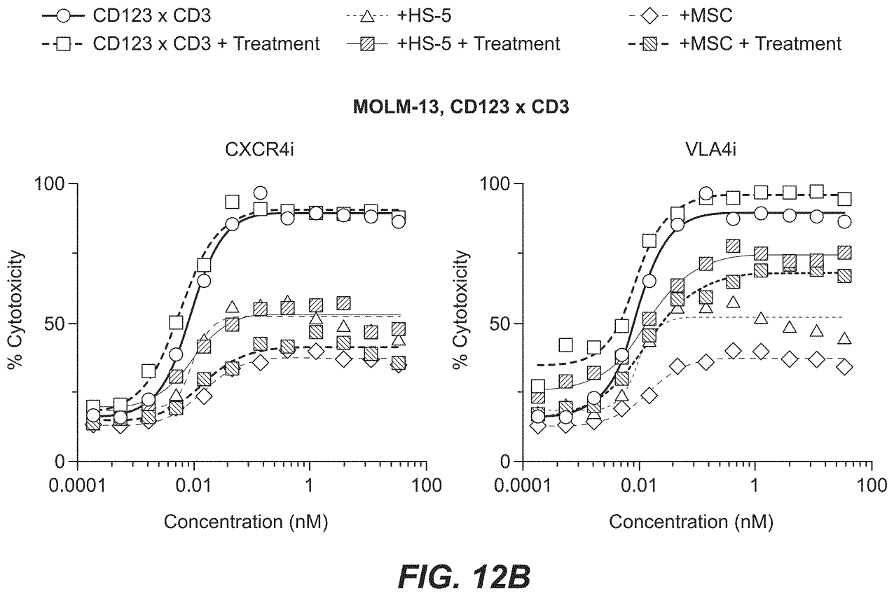

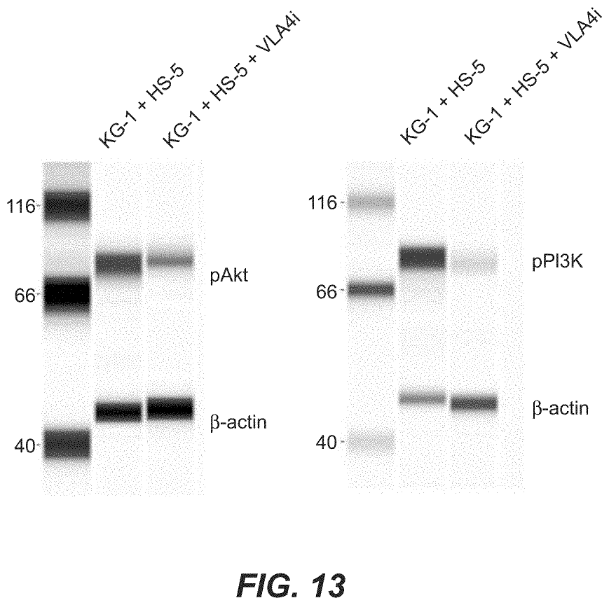

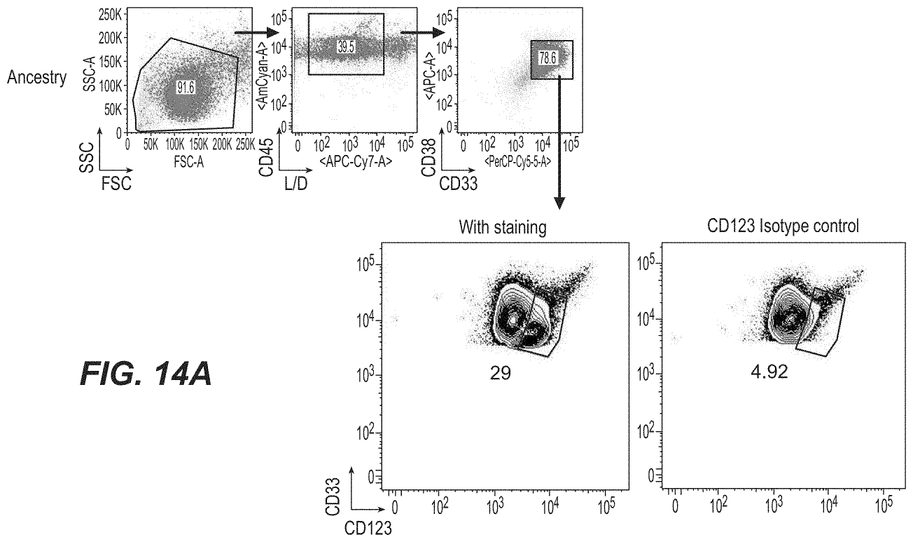

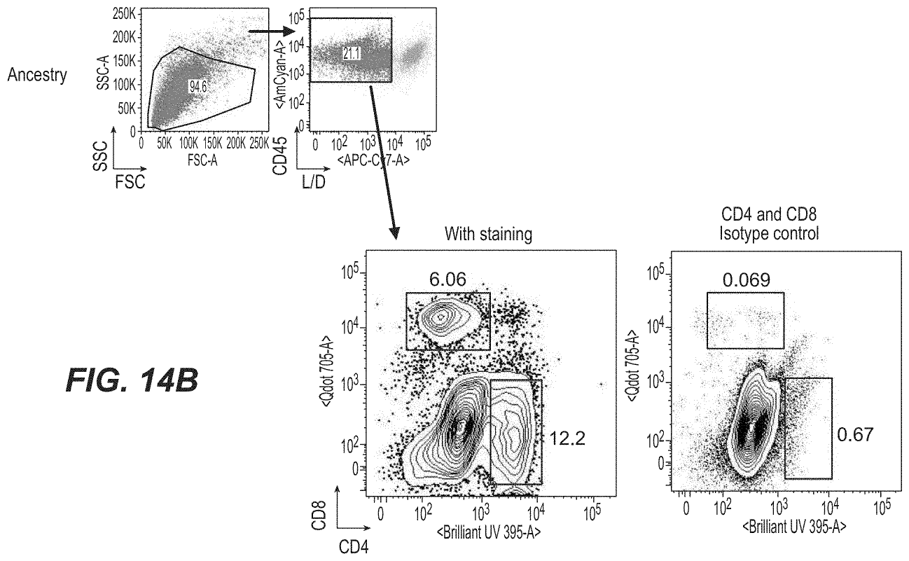

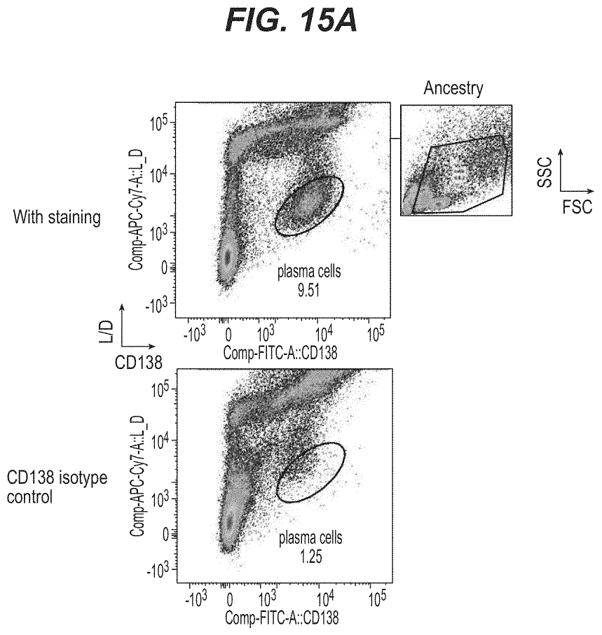

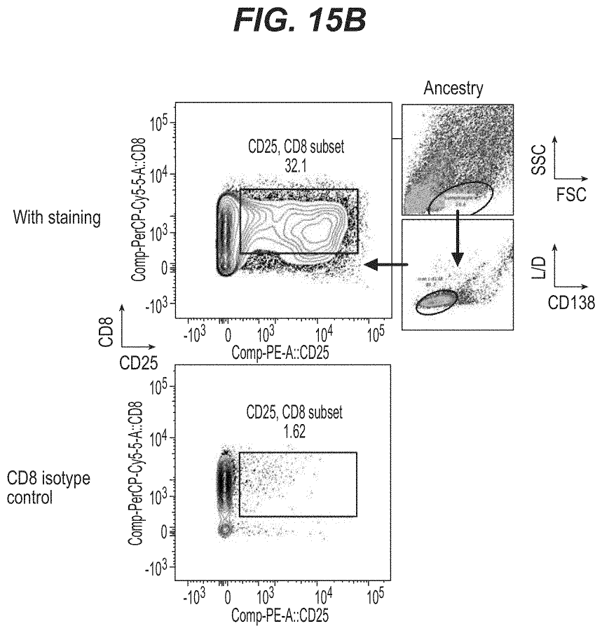

PBS-treated groups were included as controls. FIG. 3A: Mean tumor volume measurements for all the groups at different time points. FIG. 3B: Percentage of CD8⁺ T cell infiltration in the tumors of mice at the end of the study (day 24). FIG. 3C: Analysis of activation, effector and checkpoint inhibition markers in CD8⁺ T cells in the tumors of mice on day 24. All data shown are representative of two independent experiments and are represented as either mean±standard error of mean (SEM) (FIG. 3A) or median with range (FIGS. 3B and 3C). * p<0.05, ** p<0.005, *** p<0.0005; n.s., not statistically significant.

FIGS. 4A-4C show that cell-cell contact plays a dominant role in mediating the immune-suppressive and protective phenotype of stromal cells. FIG. 4A: Human T cells (40,000 cells/well) were cultured with Incucyte NucLight® Red labelled OCI-AML5 cells (20,000) with or without Incucyte NucLight® Green labelled HS-5 cells (20,000 cells per well) and were treated with varying concentrations of bispecific antibody for 72 hours. Representative images show a snapshot of the cultures at 72 hours post addition of 11 nM of CD123×CD3 bispecific antibody. FIG. 4B: Same as A but here the T cells were cultured with CFSE labelled tumor cells with or without stromal cells (HS-5 or primary MSC) and were treated with varying concentrations of bispecific antibody for 48 hours. In these assays, stromal cells were either cultured together or separated from the tumor and T cells in a trans-well. The percentage of dead CFSE⁺ cells was quantitated by flow cytometry. Data from one experiment shown here which is representative for 3 independent biological repeats. Data shown here as mean±SD. FIG. 4C: Flow analysis of activation and effector markers on CD8⁺ T cells in the killing assays. Data shown as median with range. * p<0.05, ** p<0.005, *** p<0.0005; n.s., not statistically significant.

FIGS. 5A-5D show that VLA-4 inhibition reverses stromal-mediated immune-suppression and protection of tumor cells from CD3 redirected cytotoxicity in vitro. Human T cells were cultured with CFSE labelled tumor cells with or without stromal cells (HS-5 or primary MSC) and were treated with varying concentrations of bispecific antibody for 48 hours in the presence or absence of neutralizing antibodies to VLA-4 or CXCR4. FIGS. 5A-5B: The percentage of dead CFSE⁺ cells was quantitated by flow cytometry. FIGS. 5C-5D: Flow analysis of granzyme B and CD25 expression on CD8⁺ T cells in the killing assays. Data are representative of three or more experiments and are represented as mean±SD (FIG. 5A, FIG. 5B) and median with range (FIG. 5C, FIG. 5D). * p<0.05, ** p<0.005, *** p<0.0005; **** p<0.0001; n.s., not statistically significant.

FIGS. 6A-6B show that VLA-4 inhibition reverses stromal-mediated immune-suppression and protection of tumor cells from CD3 redirected cytotoxicity in vivo. AML cell line MOLM-13 and MOLM-13 with HS-5 bone marrow stromal cells (5:1) were implanted sc in huPBMC injected female NSG mice on study day 0. Mice were treated with CD123×CD3 (8 μg/kg) either alone or in combination with a neutralizing antibody against VLA-4 (3 mg/kg). PBS treated groups were included as controls. FIG. 6A: Mean tumor volume measurements for all the groups at different time points. FIG. 6B: Analysis of activation, effector and checkpoint inhibition markers in CD8⁺ T cells in the tumors of mice on day 23. All data shown are representative of two independent experiments and are represented as mean±SEM (FIG. 6A) and median with range (FIG. 6B). * p<0.05, ** p<0.005, *** p<0.0005; **** p<0.0001; n.s., not statistically significant.

FIGS. 7A-7D show that VLA-4 inhibition rescues efficacy of CD3 redirection in ex vivo primary AML and MM cultures. PBMCs from 3 primary AML samples (FIG. 7A, FIG. 7B) or BMMCs from 3 MM samples (FIG. 7C, FIG. 7D) were incubated with bispecific antibodies at 1 μg/mL in the presence of HS-5 and with/without neutralizing antibodies against VLA-4 for 72 hours. Median values with range are depicted for cytotoxicity (FIGS. 7A and 7C) or CD8 T cell expansion (FIG. 7B)/activation (FIG. 7D) for all 3 primary samples. * p<0.05.

FIGS. 8A-8B show that CD123×CD3 and BCMA×CD3 bind tumor cells as well as mediate killing and T cell activation. FIG. 8A: CD123⁺ or BCMA⁺ cell lines were stained with various concentrations of the bispecific antibodies to characterize the surface binding profiles. Binding of the bispecific antibody was detected by staining with mouse anti-human IgG4. FIG. 8B: Ability of CD123×CD3 and BCMA×CD3 to mediate T cell activation (measured by CD25 upregulation and production of granzyme b) and cytotoxicity of CD123⁺ or BCMA⁺ tumor cell lines.

FIGS. 9A-9D show that the presence of stromal cells protects AML and MM cell lines from T cell redirected cytotoxicity. Human T cells were cultured with CFSE labelled AML or MM cell lines at a 2:1 ratio in the presence or absence of stromal cells (HS-5, HS-27a, primary MSC or CD105⁺ endothelial cells). Varying concentrations of CD123×CD3 or BCMA×CD3 were added to cultures for 48 hours. The percentage of dead CFSE⁺ cells was quantitated by flow cytometry. FIG. 9A: Table showing a summary of the EC50 values of CD123×CD3 and BCMA×CD3 to mediate cytotoxicity of AML cell line KG-1 and MM cell line H929, respectively. FIG. 9B: Similar experimental setup to A but here increasing amounts of HS-5 cells were added to KG-1-T cell cultures. FIG. 9C: Ability of CD123×CD3 to mediate cytotoxicity of AML cell lines OCI-AML5 and MOLM-13, in the absence or presence of stroma was assessed. FIG. 9D: Ability of BCMA×CD3 to mediate cytotoxicity of MM cell lines RPMI-8226 and MM.1S, in the absence or presence of stroma was assessed.

FIGS. 10A-10B show that the presence of stromal cells dampens T cell activation and proliferation. FIG. 10A: Human T cells were cultured with CFSE labelled MM cell line H929 at a 2:1 ratio in the presence or absence of stromal cells (HS-5, HS-27a, primary MSC or CD105+ endothelial cells). Varying concentrations of BCMA×CD3 were added to cultures for 48 hours. Geometric mean fluorescence intensities of T cell activation markers were quantified by flow cytometry at 48 hours post-treatment with bispecific antibodies. FIG. 10B: Similar to A but here CFSE-labelled T cells were cultures with unlabelled tumor cell lines at a 2:1 ratio in the presence or absence of stromal cells (HS-5, primary MSC or CD105⁺ endothelial cells). FACS analyses showing overlay of CFSE dilution profiles (null×CD3 control shown in shaded while treatment group shown in black histograms), depicting T cell proliferation.

FIG. 11 shows that treatment with Bcl-2 inhibitor blocks expression of Bcl2. Immunoblotting analysis of expression of Bcl-2 in KG-1 cells cultured in the presence of HS5 stromal cell line and treated with or without Bcl-2i for 48 hours.

FIGS. 12A-12B show that cell-cell contact and the VLA-4 adhesion pathway plays a role in the stromal mediated suppression of cytotoxicity in MOLM-13 cells. FIG. 12A: Human T cells were cultured with CFSE labelled MOLM-13 cells with or without HS-5 or primary MSC cells and were treated with varying concentrations of bispecific antibody for 48 hours. In these assays, stromal cells were either cultured together or separated from the tumor and T cells in a trans-well. The percentage of dead CFSE⁺ cells was quantitated by flow cytometry. FIG. 12B: Similar to A but here cytotoxicity was assessed when all cells were cultured together and in the presence or absence of neutralizing antibodies to VLA-4 or CXCR4.

FIG. 13 shows that treatment with VLA-4 neutralizing antibody reduces phosphorylation of AKT and PI3K pathways. Immunoblotting analysis of expression of pAkt and pPI3K in KG-1 cells cultured in the presence of HS5 stromal cell line and treated with or without anti-VLA4 neutralizing antibody for 48 hours.

FIGS. 14A-14B show the gating strategy for the AML primary patient samples. Gating strategy for the ex vivo experiments was conducted with primary AML patient samples. The corresponding isotype controls are shown on the side. FIG. 14A: CD123⁺ blasts were identified by first gating on forward scatter (FSC) and side scatter (SSC) to isolate cells of interest. Live CD45⁺ cells were then gated on, after which CD38⁺ CD33⁺ blasts were gated on. Next, blasts expressing CD123 were quantified in the various conditions. FIG. 14B: To quantify T cell expansion in the samples, cells of interest with SSC/FSC was first gated and then live CD45⁺ cells were identified. Then, CD4⁺CD8⁻ and CD8⁺CD4⁻ T cells were identified based on CD4 and CD8 staining.

FIGS. 15A-15B show the gating strategy for the MM primary patient samples. Gating strategy for the ex vivo experiments was conducted with primary MM patient samples. The corresponding isotype controls are shown on the side. FIG. 15A: CD138⁺ MM cells were identified by first gating on FSC/SSC to isolate cells of interest. Live CD138⁺ cells were then quantified in the various conditions. FIG. 15B: To quantify the T cell activation in the samples, lymphocytes with SSC/FSC were first gated and then live CD138⁻ cells were identified. Then, the expression of CD25 on CD8⁺ T cells were measured.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

The words “right,” “left,” “lower,” and “upper” designate directions in the drawings to which reference is made.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

As used herein, the term “isolated” means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if the composition is not part of the native environment of the nucleic acid, peptide, or protein. The term also embraces nucleic acids, peptides and proteins prepared by recombinant expression in a host cell as well as chemically synthesized nucleic acids.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA.

“Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule of the invention. The “host cell” can be any type of cell, e.g., a primary cell, a cell in culture, or a cell from a cell line. In one embodiment, a “host cell” is a cell transfected or transduced with a nucleic acid molecule of the invention. In another embodiment, a “host cell” is a progeny or potential progeny of such a transfected or transduced cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications.

The term “antibodies” as used herein, is meant in a broad sense and includes immunoglobulin molecules including monoclonal antibodies (including murine, human, humanized and chimeric monoclonal antibodies), antigen binding fragments, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g. IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4 Immunoglobulins may be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains.

The term “monoclonal antibody” as used herein, refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation. Monoclonal antibodies typically bind one antigenic epitope. A bispecific monoclonal antibody binds two distinct antigenic epitopes. Monoclonal antibodies may have heterogeneous glycosylation within the antibody population. Monoclonal antibody may be monospecific or multispecific such as bispecific, monovalent, bivalent or multivalent.

The term “human antibody” as used herein, refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” may contain consensus framework sequences derived from human framework sequence analyses, for example as described in Knappik et al., (2000) J Mol Biol 296:57-86, or synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

The term “humanized antibody” as used herein, refers to an antibody in which at least one CDR is derived from non-human species and at least one framework is derived from human immunoglobulin sequences. Humanized antibody may include substitutions in the frameworks so that the frameworks may not be exact copies of expressed human immunoglobulin or human immunoglobulin germline gene sequences.

The term “isolated antibody” refers to an antibody that is substantially free of other cellular material and/or chemicals and encompasses antibodies that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

The term “antigen binding fragment” or “antigen binding domain” as used herein, refers to a portion of an immunoglobulin molecule that binds an antigen. Antigen binding fragments may be synthetic, enzymatically obtainable or genetically engineered polypeptides and include the VH, the VL, the VH and the VL, Fab, F(ab′)2, Fd and Fv fragments, domain antibodies (dAb) consisting of one VH domain or one VL domain, shark variable IgNAR domains, camelized VH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3. VH and VL domains may be linked together via a synthetic linker to form various types of single chain antibody designs where the VH/VL domains may pair intramolecularly, or intermolecularly in those cases when the VH and VL domains are expressed by separate single chain antibody constructs, to form a monovalent antigen binding site, such as single chain Fv (scFv) or diabody; described for example in Int. Patent Publ. Nos. WO1998/44001, WO1988/01649, WO1994/13804 and WO1992/01047.

The term “bispecific” refers to an antibody that specifically binds two distinct antigens or two distinct epitopes within the same antigen. The bispecific antibody may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

The term “multispecific” as used herein, refers to an antibody that specifically binds at least two distinct antigens or at least two distinct epitopes within the same antigen. Multispecific antibody may bind for example two, three, four or five distinct antigens or distinct epitopes within the same antigen.

“Specific binding” or “immunospecific binding” or derivatives thereof when used in the context of antibodies, or antibody fragments, represents binding via domains encoded by immunoglobulin genes or fragments of immunoglobulin genes to one or more epitopes of a protein of interest, without preferentially binding other molecules in a sample containing a mixed population of molecules. Typically, an antibody binds to a cognate antigen with a K_{d} of less than about 1×10⁻⁸M, as measured by a surface plasmon resonance assay or a cell binding assay.

The term “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor.

The term “combination” as used herein, means that two or more therapeutics are administered to a subject together in a mixture, concurrently as single agents or sequentially as single agents in any order.

The term “enhance” or “enhanced” as used herein, refers to enhancement in one or more functions of a test molecule when compared to a control molecule or a combination of test molecules when compared to one or more control molecules. Exemplary functions that can be measured are tumor cell killing, T cell activation, relative or absolute T cell number, Fc-mediated effector function (e.g. ADCC, CDC and/or ADCP) or binding to an Fcγ receptor (FcγR) or FcRn. “Enhanced” may be an enhancement of about 10%, 20%, 30%, 40%, 50%, 60%, 70%, 80%, 90%, 100% or more, or a statistically significant enhancement.

The term “mutation” as used herein, refers to an engineered or naturally occurring alteration in a polypeptide or polynucleotide sequence when compared to a reference sequence. The alteration may be a substitution, insertion or deletion of one or more amino acids or polynucleotides.

The term “non-fixed combination” as used herein, refers to separate pharmaceutical compositions of the T cell redirection therapeutic and the VLA-4 adhesion pathway inhibitor administered as separate entities either simultaneously, concurrently or sequentially with no specific intervening time limits, wherein such administration provides effective levels of the two compounds in the body of the subject.

The term “pharmaceutical composition” as used herein, refers to composition that comprises an active ingredient and a pharmaceutically acceptable carrier.

The term “pharmaceutically acceptable carrier” or “excipient” as used herein, refers to an ingredient in a pharmaceutical composition, other than the active ingredient, which is nontoxic to a subject.

The term “recombinant” as used herein, refers to DNA, antibodies and other proteins that are prepared, expressed, created or isolated by recombinant means when segments from different sources are joined to produce recombinant DNA, antibodies or proteins.

The term “reduce” or “reduced” as used herein, refers to a reduction in one or more functions of a test molecule when compared to a control molecule or a combination of test molecules when compared to one or more control molecules. Exemplary functions that can be measured are tumor cell killing, T cell activation, relative or absolute T cell number, Fc-mediated effector function (e.g. ADCC, CDC and/or ADCP) or binding to an Fcγ receptor (FcγR) or FcRn. “Reduced” may be a reduction of about 10%, 20%, 30%, 40%, 50%, 60%, 70%, 80%, 90%, 100% or more, or a statistically significant enhancement.

The term “refractory” as used herein, refers to a cancer that is not amendable to surgical intervention and is initially unresponsive to therapy.

The term “relapsed” as used herein, refers to a cancer that responded to treatment but then returns.

The term “subject” as used herein, includes any human or nonhuman animal “Nonhuman animal” includes all vertebrates, e.g., mammals and non-mammals, such as nonhuman primates, sheep, dogs, cats, horses, cows, chickens, amphibians, reptiles, etc. Except when noted, the terms “patient” or “subject” are used interchangeably.

The term “therapeutically effective amount” as used herein, refers to an amount effective, at doses and for periods of time necessary, to achieve a desired therapeutic result. A therapeutically effective amount may vary depending on factors such as the disease state, age, sex, and weight of the individual, and the ability of a therapeutic or a combination of therapeutics to elicit a desired response in the individual. Exemplary indicators of an effective therapeutic or combination of therapeutics that include, for example, improved well-being of the patient.

The term “treat” or “treatment” as used herein, refers to both therapeutic treatment and prophylactic or preventative measures, wherein the object is to prevent or slow down (lessen) an undesired physiological change or disorder. Beneficial or desired clinical results include alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if a subject was not receiving treatment. Those in need of treatment include those already with the condition or disorder as well as those prone to have the condition or disorder or those in which the condition or disorder is to be prevented.

The term “tumor cell” or a “cancer cell” as used herein, refers to a cancerous, pre-cancerous or transformed cell, either in vivo, ex vivo, or in tissue culture, that has spontaneous or induced phenotypic changes. These changes do not necessarily involve the uptake of new genetic material. Although transformation may arise from infection with a transforming virus and incorporation of new genomic nucleic acid, uptake of exogenous nucleic acid or it can also arise spontaneously or following exposure to a carcinogen, thereby mutating an endogenous gene. Transformation/cancer is exemplified by morphological changes, immortalization of cells, aberrant growth control, foci formation, proliferation, malignancy, modulation of tumor specific marker levels, invasiveness, tumor growth in suitable animal hosts such as nude mice, and the like, in vitro, in vivo, and ex vivo.

### T Cell Redirection Therapeutics

The T cell redirection therapeutic (which is also referred to as “T cell redirection bispecific antibody” or “the bispecific antibody” throughout this application) disclosed herein is a molecule containing two or more binding regions, wherein one of the binding regions specifically binds a cell surface antigen (such as a tumor associated antigen (TAA)) on a target cell or tissue and wherein a second binding region of the molecule specifically binds a T cell surface antigen (such as, CD3). This dual/multi-target binding ability recruits T cells to the target cell or tissue leading to the eradication of the target cell or tissue.

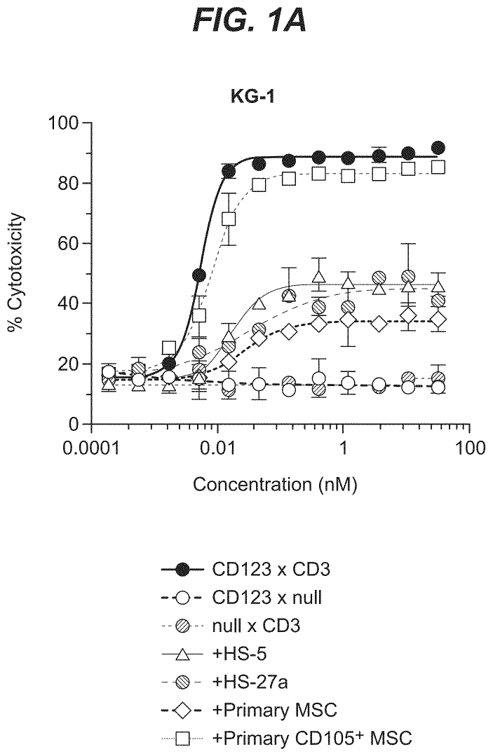

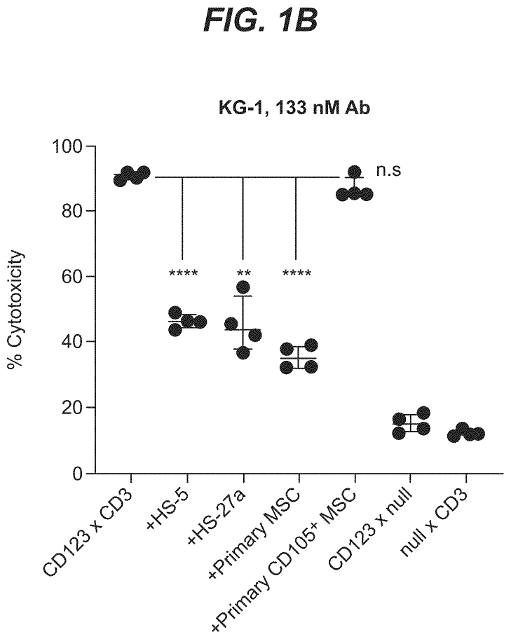

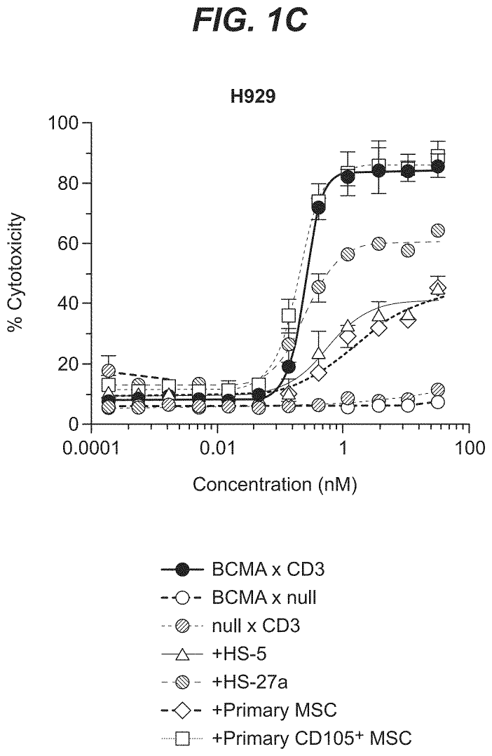

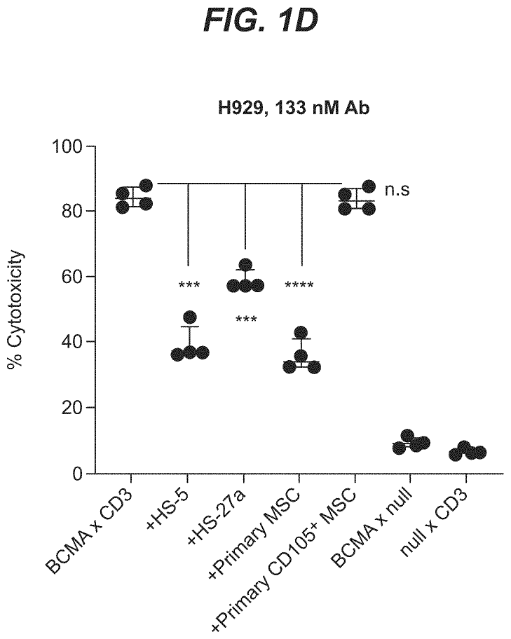

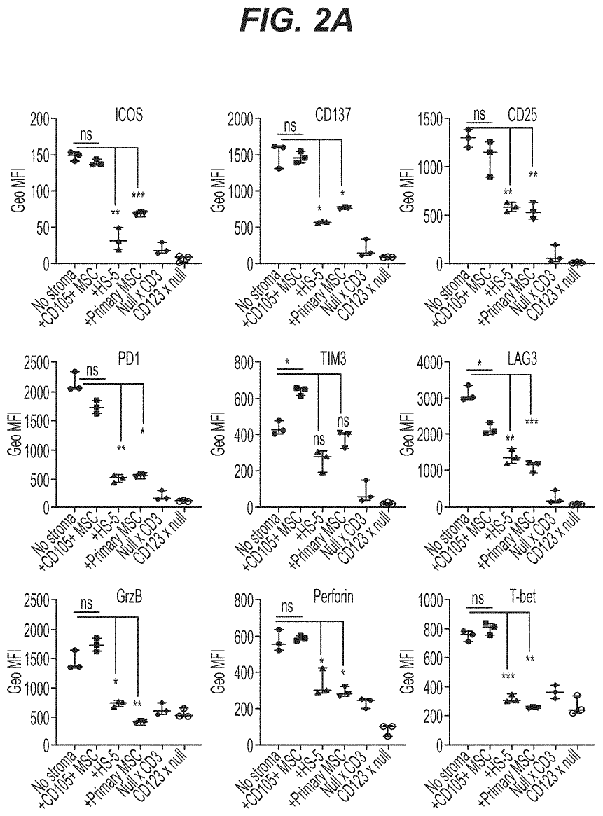

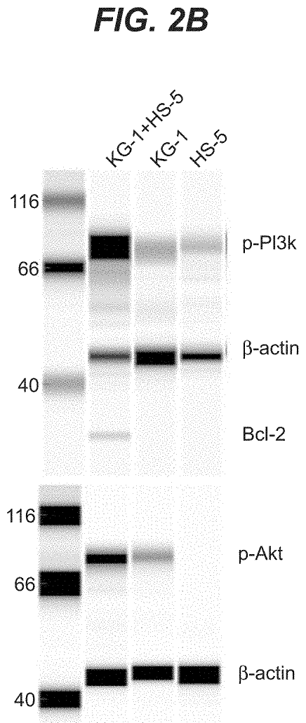

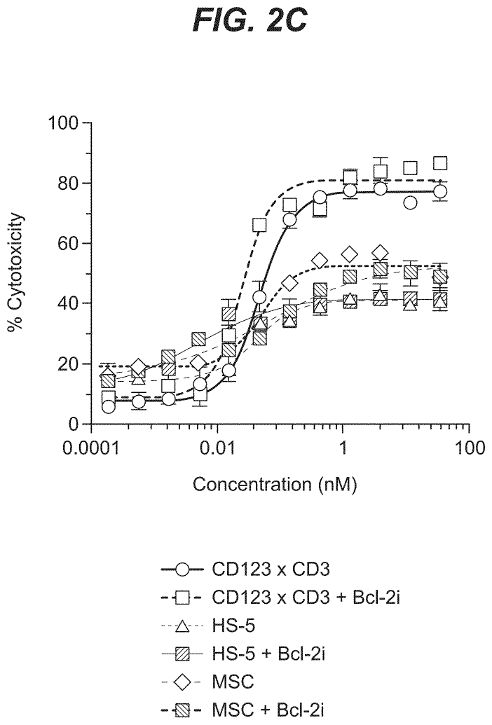

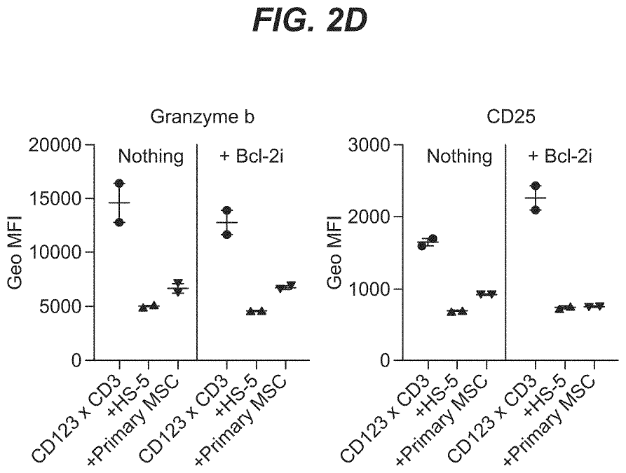

The T cell redirection therapeutic used herein may be an antibody, an antibody-derived protein, or, for example, a recombinant protein exhibiting antigen binding sites. In one embodiment, the T cell redirection therapeutics used herein are bispecific antibodies encompass “whole” antibodies, such as whole IgG or IgG-like molecules, and small recombinant formats, such as tandem single chain variable fragment molecules (taFvs), diabodies (Dbs), single chain diabodies (scDbs) and various other derivatives of these (cf. bispecific antibody formats as described by Byrne H. et al. (2013) Trends Biotech, 31 (11): 621-632 with FIG. 2 showing various bispecific antibody formats; Weidle U. H. et al. (2013) Cancer Genomics and Proteomics 10: 1-18, in particular FIG. 1 showing various bispecific antibody formats; and Chan, A. C. and Carter, P. J. (2010) Nat Rev Immu 10: 301-316 with FIG. 3 showing various bispecific antibody formats). Examples of bispecific antibody formats include, but are not limited to, quadroma, chemically coupled Fab (fragment antigen binding), and BiTE® (bispecific T cell engager).

In one embodiment, the bispecific antibody used herein may be selected from the group comprising Triomabs; hybrid hybridoma (quadroma); Multispecific anticalin platform (Pieris); Diabodies; Single chain diabodies; Tandem single chain Fv fragments; TandAbs, Trispecific Abs (Affimed) (105-110 kDa); Darts (dual affinity retargeting; Macrogenics); Bispecific Xmabs (Xencor); Bispecific T cell engagers (Bites; Amgen; 55 kDa); Triplebodies; Tribody=Fab-scFv Fusion Protein (CreativeBiolabs) multifunctional recombinant antibody derivates (110 kDa); Duobody platform (Genmab); Dock and lock platform; Knob into hole (KIH) platform; Humanized bispecific IgG antibody (REGN1979) (Regeneron); Mab2 bispecific antibodies (F-Star); DVD-Ig=dual variable domain immunoglobulin (Abbvie); kappa-lambda bodies; tetravalent bispecific tandem Ig; and CrossMab.

In a further embodiment, the bispecific antibodies as used herein may be selected from bispecific IgG-like antibodies (BsIgG) comprising CrossMab; DAF (two-in-one); DAF (four-in-one); DutaMab; DT-IgG; Knobs-in-holes common LC; Knobs-in-holes assembly; Charge pair; Fab-arm exchange; SEEDbody; Triomab; LUZ-Y; Fcab; κλ-body; and Orthogonal Fab. These bispecific antibody formats are shown and described for example in Spiess C., Zhai Q. and Carter P. J. (2015) Molecular Immunology 67: 95-106, in particular FIG. 1 and corresponding description, e.g. p. 95-101.

In yet a further embodiment, the bispecific antibodies used herein may be selected from IgG-appended antibodies with an additional antigen-binding moiety comprising DVD-IgG; IgG(H)-scFv; scFv-(H)IgG; IgG(L)-scFv; scFV-(L)IgG; IgG(L,H)-Fv; IgG(H)-V; V(H)—IgG; IgG(L)-V; V(L)-IgG; KIH IgG-scFab; 2scFv-IgG; IgG-2scFv; scFv4-Ig; scFv4-Ig; Zybody; and DVI-IgG (four-in-one). These bispecific antibody formats are shown and described for example in Spiess C., Zhai Q. and Carter P. J. (2015) Molecular Immunology 67: 95-106, in particular FIG. 1 and corresponding description, e.g. p. 95-101.

In a yet further embodiment, the bispecific antibodies used herein may be selected from bispecific antibody fragments comprising Nanobody; Nanobody-HAS; BiTE; Diabody; DART; TandAb; scDiabody; sc-Diabody-CH3; Diabody-CH3; Triple Body; Miniantibody; Minibody; TriBi minibody; scFv-CH3 KIH; Fab-scFv; scFv-CH-CL-scFv; F(ab′)2; F(ab′)2-scFv2; scFv-KIH; Fab-scFv-Fc; Tetravalent HCAb; scDiabody-Fc; Diabody-Fc; Tandem scFv-Fc; and Intrabody. These bispecific antibody formats are shown and described for example in Spiess C., Zhai Q. and Carter P. J. (2015) Molecular Immunology 67: 95-106, in particular FIG. 1 and corresponding description, e.g. p. 95-101.

In a yet further embodiment, the bispecific antibodies used herein may be selected from bispecific fusion proteins comprising Dock and Lock; ImmTAC; HSAbody; scDiabody-HAS; and Tandem scFv-Toxin. These bispecific antibody formats are shown and described for example in Spiess C., Zhai Q. and Carter P. J. (2015) Molecular Immunology 67: 95-106, in particular FIG. 1 and corresponding description, e.g. p. 95-101.

In a yet further embodiment, the bispecific antibodies used herein may be selected from bispecific antibody conjugates comprising IgG-IgG; Cov-X-Body; and scFv1-PEG-scFv2. These bispecific antibody formats are shown and described for example in Spiess C., Zhai Q. and Carter P. J. (2015) Molecular Immunology 67: 95-106, in particular FIG. 1 and corresponding description, e.g. p. 95-101.

In yet further embodiment, the bispecific antibodies used herein may be based on any immunoglobulin class (e.g., IgA, IgG, IgM etc.) and subclass (e.g. IgA1, IgA2, IgG1, IgG2, IgG3, IgG4 etc.). In aspect, the bispecific antibodies used herein may have an IgG-like format (based on IgG, also referred to as “IgG type”), which usually comprises two heavy chains and two light chains. Examples of antibodies having an IgG-like format include a quadroma and various IgG-scFv formats (cf: Byrne H. et al. (2013) Trends Biotech, 31 (11): 621-632; FIG. 2A-E), whereby a quadroma is preferred, which is preferably generated by fusion of two different hybridomas. Within the IgG class, the bispecific antibodies may be based on the IgG1, IgG2, IgG3 or IgG4 subclass.

In yet a further embodiment, the bispecific antibodies used herein are in IgG-like antibody formats, which comprise for example hybrid hybridoma (quadroma), knobs-into-holes with common light chain, various IgG-scFv formats, various scFv-IgG formats, two-in-one IgG, dual V domain IgG, IgG-V, and V-IgG, which are shown for example in FIG. 3c of Chan, A. C. and Carter, P. J. (2010) Nat Rev Immu 10: 301-316 and described in said article. Further exemplary bispecific IgG-like antibody formats include for example DAF, CrossMab, IgG-dsscFv, DVD, IgG-dsFV, IgG-scFab, scFab-dsscFv and Fv2-Fc, which are shown in FIG. 1A of Weidle U. H. et al. (2013) Cancer Genomics and Proteomics 10: 1-18 and described in said article. Yet further exemplary bispecific IgG-like antibody formats include DAF (two-in-one); DAF (four-in-one); DutaMab; DT-IgG; Knobs-in-holes assembly; Charge pair; Fab-arm exchange; SEEDbody; Triomab; LUZ-Y; Fcab; κλ-body; Orthogonal Fab; DVD-IgG; IgG(H)-scFv; scFv-(H)IgG; IgG(L)-scFv; scFV-(L)IgG; IgG(L,H)-Fv; IgG(H)-V; V(H)-IgG; IgG(L)-V; V(L)-IgG; KIH IgG-scFab; 2scFv-IgG; IgG-2scFv; scFv4-Ig; scFv4-Ig; Zybody; and DVI-IgG (four-in-one) as shown and described for example in Spiess C., Zhai Q. and Carter P. J. (2015) Molecular Immunology 67: 95-106, in particular FIG. 1 and corresponding description, e.g. p. 95-101.

Bispecific antibodies, for example, can be produced by three different methods: (i) chemical conjugation, which involves chemical cross-linking; (ii) fusion of two different hybridoma cell lines; or (iii) genetic approaches involving recombinant DNA technology. The fusion of two different hybridomas produces a hybrid-hybridoma (or “quadroma”) secreting a heterogeneous antibody population including bispecific molecules. Alternative approaches included chemical conjugation of two different mAbs and/or smaller antibody fragments. Oxidative reassociation strategies to link two different antibodies or antibody fragments were found to be inefficient due to the presence of side reactions during reoxidation of the multiple native disulfide bonds. Current methods for chemical conjugation focus on the use of homo- or hetero-bifunctional crosslinking reagents. Recombinant DNA technology has yielded the greatest range of bispecific antibodies, through artificial manipulation of genes and represents the most diverse approach for bispecific antibody generation (45 formats in the past two decades; cf. Byrne H. et al. (2013) Trends Biotech, 31 (11): 621-632).

In particular by use of such recombinant DNA technology, also a variety of further multispecific antibodies have emerged recently. The term “multispecific antibodies” refers to proteins having more than one paratope and the ability to bind to two or more different epitopes. Thus, the term “multispecific antibodies” comprises bispecific antibodies as defined above, but typically also protein, e.g. antibody, scaffolds, which bind in particular to three or more different epitopes, i.e. antibodies with three or more paratopes. Such multispecific proteins, in particular with three or more paratopes, are typically achieved by recombinant DNA techniques. In the context of the present invention, the antibody may in particular also have more than two specificities, and, thus, more than two paratopes, as at least two paratopes are required according to the present invention, for example one for the target cell and the other for a T cell. Accordingly, the antibody to be used according to the invention may have further paratopes, in particular relating to further specificities, in addition to the two paratopes. Thus, the present invention also comprises multispecific antibodies. It is thus understood that the invention is not limited to bispecific antibodies, although it is referred herein in particular to bispecific antibodies, which represent the minimum requirements. What is said herein about bispecific antibodies may therefore also apply to multispecific antibodies.

The bispecific antibodies, and multispecific antibodies as defined above, are able to redirect effector cells against target cells that play key roles in disease processes. In particular, the T cell redirection bispecific antibodies used herein can, for example, bind to T cell receptor (TCR) complexes and “redirect” T cells to target cells, such as for example tumor cells. To this end, such bispecific antibodies used herein typically has at least one specificity, e.g. at least one paratope, for recruiting T cells, which is specific for T cells, preferably for T cell surface antigens, e.g. CD3, and at least one other specificity, e.g. at least one paratope, for directing T cells to tumor cells, which is specific for tumor cells, preferably a TAA on tumor cells. Such a “redirection” of a T cell to a tumor cell by a T cell redirection bispecific antibody typically results in T-cell mediated cell killing of the tumor cell.

In one embodiment, the T cell redirection therapeutic used herein comprise a first binding region with specificity against a T cell surface antigen and a second binding region with specificity against a TAA on a tumor cell.

In a further embodiment, the T cell surface antigen may be selected from CD3, CD2, CD4, CD5, CD6, CD8, CD28, CD40L, CD44, CD137, KI2L4, NKG2E, NKG2D, NKG2F, BTNL3, CD186, BTNL8, PD-1, CD195, and NKG2C. Or, the T cell surface antigen is CD3.

In a yet further embodiment, the TAA may be selected from B-cell maturation antigen (BCMA), CD123, GPRC5D, CD33, CD19, PSMA, TMEFF2, CD20, CD10, CD21, CD22, CD25, CD30, CD34, CD37, CD44v6, CD45, CD52, CD133, ROR1, B7-H6, B7-H3, HM1.24, SLAMF7, Fms-like tyrosine kinase 3 (FLT-3, CD135), chondroitin sulfate proteoglycan 4 (CSPG4, melanoma-associated chondroitin sulfate proteoglycan), epidermal growth factor receptor (EGFR), Her2, Her3, IGFR, IL3R, fibroblast activating protein (FAP), CDCP1, Derlin1, Tenascin, frizzled 1-10, VEGFR2 (KDR/FLK1), VEGFR3 (FLT4, CD309), PDGFR-alpha (CD140a), PDGFR-beta (CD140b), endoglin, CLEC14, Tem1-8, or Tie2. Further exemplary TAA on the tumor cell include A33, CAMPATH-1 (CDw52), Carcinoembryonic antigen (CEA), Carboanhydrase IX (MN/CA IX), de2-7, EGFRvIII, EpCAM, Ep-CAM, folate-binding protein, G250, c-Kit (CD117), CSF1R (CD115), HLA-DR, IGFR, IL-2 receptor, IL3R, MCSP (melanoma-associated cell surface chondroitin sulphate proteoglycane), Muc-1, prostate stem cell antigen (PSCA), prostate specific antigen (PSA), hK2, TAG-72 or a tumor cell neoantigen. Or, the TAA may be selected from BCMA, CD123, GPRC5D, CD33, CD19, PSMA, TMEFF2, CD20, CD22, CD25, CD52, ROR1, HM1.24, CD38 and SLAMF7. Or, the TAA may be selected from BCMA or CD123.

In one embodiment, the T cell redirection therapeutic is a BCMAxCD3 bispecific antibody that immunospecifically binds to BCMA⁺ MM cells and CD3 T cells. The BCMAxCD3 bispecific antibodies may be selected from those disclosed in WO2007117600, WO2009132058, WO2012066058, WO2012143498, WO2013072406, WO2013072415, WO2014122144, and U.S. Pat. No. 10,072,088, which are incorporated herein by reference in their entirety.

In one embodiment, the BCMAxCD3 bispecific antibody is a bispecific DuoBody® antibody as those disclosed in U.S. Pat. No. 10,072,088, which is incorporated herein by reference in its entirety. The BCMAxCD3 bispecific antibody comprises a first heavy chain (HC1), a first light chain (LC1), a second heavy chain (HC2), and a second light chain (LC2), in which HC1 and LC1 pair to form a first antigen-binding site that immunospecifically binds BCMA, and HC2 and LC2 pair to form a second antigen-binding site that immunospecifically binds CD3. In one embodiment, the BCMAxCD3 antibody comprises HC1 having the amino acid sequence of SEQ ID NO: 1, LC1 having the amino acid sequence of SEQ ID NO: 2, HC2 having the amino acid sequence of SEQ ID NO: 3, and LC2 having the amino acid sequence of SEQ ID NO: 4, wherein HC1 and LC1 pair to form a first antigen-binding site that immunospecifically binds BCMA, and HC2 and LC2 pair to form a second antigen-binding site that immunospecifically binds CD3. In one embodiment, the BCMAxCD3 antibody comprises HC1 having the amino acid sequence of SEQ ID NO: 5, LC1 having the amino acid sequence of SEQ ID NO: 6, HC2 having the amino acid sequence of SEQ ID NO: 3, and LC2 having the amino acid sequence of SEQ ID NO: 4, wherein HC1 and LC1 pair to form a first antigen-binding site that immunospecifically binds BCMA, and HC2 and LC2 pair to form a second antigen-binding site that immunospecifically binds CD3.

In one embodiment, the T cell redirection therapeutic is a CD123×CD3 bispecific antibody that immunospecifically binds to CD123⁺ AML cells and CD3 T cells. The CD123×CD3 bispecific antibody may be a bispecific DuoBody® antibody as those disclosed in U.S. Pat. No. 9,850,310, which is incorporated herein by reference in its entirety. In one embodiment, the CD123×CD3 antibody comprises a HC1 having the amino acid sequence of SEQ ID NO: 7, a LC1 having the amino acid sequence of SEQ ID NO: 8, a HC2 having the amino acid sequence of SEQ ID NO: 9, and a LC2 having the amino acid sequence of SEQ ID NO: 10, wherein HC1 and LC1 pair to form a first antigen-binding site that immunospecifically binds CD123, and HC2 and LC2 pair to form a second antigen-binding site that immunospecifically binds CD3.

SEQ ID NO: 1
QLQLQESGPGLVKPSETLSLTCTVSGDSISKNSYYWGWIRQ
PPGKGLEWIGSMYYSGSTYYNSSLKSRVTISVDTSKNQFSL
KLSSVTAADTAVYYCARHDGGASIFDYWGQGTLVTVSSAST
KGPSVFPLAPCSRSTSESTAALGCLVKDYFPEPVTVSWNSG
ALTSGVHTFPAVLQSSGLYSLSSVVTVPSSSLGTKTYTCNV
DHKPSNTKVDKRVESKYGPPCPPCPAPEAAGGPSVFLFPPK
PKDTLMISRTPEVTCVVVDVSQEDPEVQFNWYVDGVEVHNA
KTKPREEQFNSTYRVVSVLTVLHQDWLNGKEYKCKVSNKGL
PSSIEKTISKAKGQPREPQVYTLPPSQEEMTKNQVSLTCLV
KGFYPSDIAVEWESNGQPENNYKTTPPVLDSDGSFFLYSRL
TVDKSRWQEGNVFSCSVMHEALHNHYTQKSLSLSLGK
SEQ ID NO: 2
SYELTQPPSVSVSPGQTASITCSGDKLGDMDACWYQQRPGQ
SPVVVIYQDSERPSGIPERFAGSNSGNTATLTISGTQAMDE
ADYYCQAWDSSTVVFGGGTKLTVLGQPKAAPSVTLFPPSSE
ELQANKATLVCLISDFYPGAVTVAWKGDSSPVKAGVETTTP
SKQSNNKYAASSYLSLTPEQWKSHRSYSCQVTHEGSTVEKT
VAPTECS
SEQ ID NO: 3
EVQLVESGGGLVQPGGSLRLSCAASGFTFNTYAMNWVRQAP
GKGLEWVARIRSKYNNYATYYAASVKGRFTISRDDSKNSLY
LQMNSLKTEDTAVYYCARHGNFGNSYVSWFAYWGQGTLVTV
SSASTKGPSVFPLAPCSRSTSESTAALGCLVKDYFPEPVTV
SWNSGALTSGVHTFPAVLQSSGLYSLSSVVTVPSSSLGTKT
YTCNVDHKPSNTKVDKRVESKYGPPCPPCPAPEAAGGPSVF
LFPPKPKDTLMISRTPEVTCVVVDVSQEDPEVQFNWYVDGV
EVHNAKTKPREEQFNSTYRVVSVLTVLHQDWLNGKEYKCKV
SNKGLPSSIEKTISKAKGQPREPQVYTLPPSQEEMTKNQVS
LTCLVKGFYPSDIAVEWESNGQPENNYKTTPPVLDSDGSFL
LYSKLTVDKSRWQEGNVFSCSVMHEALHNHYTQKSLSLSLG
K
SEQ ID NO: 4
QTVVTQEPSLTVSPGGTVTLTCRSSTGAVTTSNYANWVQQK
PGQAPRGLIGGTNKRAPGTPARFSGSLLGGKAALTLSGVQP
EDEAEYYCALWYSNLWVFGGGTKLTVLGQPKAAPSVTLFPP
SSEELQANKATLVCLISDFYPGAVTVAWKADSSPVKAGVET
TTPSKQSNNKYAASSYLSLTPEQWKSHRSYSCQVTHEGSTV
EKTVAPTECS
SEQ ID NO: 5
QLQLQESGPGLVKPSETLSLTCTVSGGSISSGSYFWGWIRQ
PPGKGLEWIGSIYYSGITYYNPSLKSRVTISVDTSKNQFSL
KLSSVTAADTAVYYCARHDGAVAGLFDYWGQGTLVTVSSAS
TKGPSVFPLAPCSRSTSESTAALGCLVKDYFPEPVTVSWNS
GALTSGVHTFPAVLQSSGLYSLSSVVTVPSSSLGTKTYTCN
VDHKPSNTKVDKRVESKYGPPCPPCPAPEAAGGPSVFLFPP
KPKDTLMISRTPEVTCVVVDVSQEDPEVQFNWYVDGVEVHN
AKTKPREEQFNSTYRVVSVLTVLHQDWLNGKEYKCKVSNKG
LPSSIEKTISKAKGQPREPQVYTLPPSQEEMTKNQVSLTCL
VKGFYPSDIAVEWESNGQPENNYKTTPPVLDSDGSFFLYSR
LTVDKSRWQEGNVFSCSVMHEALHNHYTQKSLSLSLGK
SEQ ID NO: 6
SYVLTQPPSVSVAPGQTARITCGGNNIGSKSVHWYQQPPGQ
APVVVVYDDSDRPSGIPERFSGSNSGNTATLTISRVEAGDE
AVYYCQVWDSSSDHVVFGGGTKLTVLGQPKAAPSVTLFPPS
SEELQANKATLVCLISDFYPGAVTVAWKGDSSPVKAGVETT
TPSKQSNNKYAASSYLSLTPEQWKSHRSYSCQVTHEGSTVE
KTVAPTECS
SEQ ID NO: 7
EVQLVQSGAEVKKPGESLKISCKGSGYSFTSYWISWVRQMP
GKGLEWMGIIDPSDSDTRYSPSFQGQVTISADKSISTAYLQ
WSSLKASDTAMYYCARGDGSTDLDYWGQGTLVTVSSASTKG
PSVFPLAPCSRSTSESTAALGCLVKDYFPEPVTVSWNSGAL
TSGVHTFPAVLQSSGLYSLSSVVTVPSSSLGTKTYTCNVDH
KPSNTKVDKRVESKYGPPCPPCPAPEAAGGPSVFLFPPKPK
DTLMISRTPEVTCVVVDVSQEDPEVQFNWYVDGVEVHNAKT
KPREEQFNSTYRVVSVLTVLHQDWLNGKEYKCKVSNKGLPS
SIEKTISKAKGQPREPQVYTLPPSQEEMTKNQVSLTCLVKG
FYPSDIAVEWESNGQPENNYKTTPPVLDSDGSFFLYSRLTV
DKSRWQEGNVFSCSVMHEALHNHYTQKSLSLSLGK
SEQ ID NO: 8
EIVLTQSPGTLSLSPGERATLSCRASQSVSSSYLAWYQQKP
GQAPRLLIYGASSRATGIPDRFSGSGSGTDFTLTISRLEPE
DFAVYYCQQDYGFPWTFGQGTKVEIKRTVAAPSVFIFPPSD
EQLKSGTASVVCLLNNFYPREAKVQWKVDNALQSGNSQESV
TEQDSKDSTYSLSSTLTLSKADYEKHKVYACEVTHQGLSSP
VTKSFNRGEC
SEQ ID NO: 9
EVQLVESGGGLVQPGGSLKLSCAASGFTFNTYAMNWVRQAS
GKGLEWVGRIRSKYNAYATYYAASVKGRFTISRDDSKNTAY
LQMNSLKTEDTAVYYCTRHGNFGNSYVSWFAYWGQGTLVTV
SSASTKGPSVFPLAPCSRSTSESTAALGCLVKDYFPEPVTV
SWNSGALTSGVHTFPAVLQSSGLYSLSSVVTVPSSSLGTKT
YTCNVDHKPSNTKVDKRVESKYGPPCPPCPAPEAAGGPSVF
LFPPKPKDTLMISRTPEVTCVVVDVSQEDPEVQFNWYVDGV
EVHNAKTKPREEQFNSTYRVVSVLTVLHQDWLNGKEYKCKV
SNKGLPSSIEKTISKAKGQPREPQVYTLPPSQEEMTKNQVS
LTCLVKGFYPSDIAVEWESNGQPENNYKTTPPVLDSDGSFL
LYSKLTVDKSRWQEGNVFSCSVMHEALHNHYTQKSLSLSLG
K
SEQ ID NO: 10
QAVVTQEPSLTVSPGGTVTLTCRSSTGAVTTSNYANWVQQK
PGQAPRGLIGGTNKRAPGTPARFSGSLLGGKAALTLSGAQP
EDEAEYYCALWYSNLWVFGGGTKLTVLGQPKAAPSVTLFPP
SSEELQANKATLVCLISDFYPGAVTVAWKADSSPVKAGVET
TTPSKQSNNKYAASSYLSLTPEQWKSHRSYSCQVTHEGSTV
EKTVAPTECS

### VLA-4 Adhesion Pathway Inhibitor

Very late antigen-4 (VLA-4), also known as called α4β1, is a member of the β1 integrin family of cell surface receptors. VLA-4 contains a α4 chain and a β1 chain and is involved in cell-cell interactions. Its expression is mainly restricted to lymphoid and myeloid cells. It is a key player in cell adhesion. Studies also have shown that VLA-4 plays an important role in mediating AML/MM-stroma interactions in BM. Vascular cell adhesion molecule-1 (VCAM-1) (expressed by osteoblasts and endothelial cells) and fibronectin (a component of the extracellular matrix) are two ligands for VLA-4.

The VLA-4 adhesion pathway inhibitors used herein may be any molecule that is capable of blocking the VLA-4 mediated adhesion pathway.

For example, the VLA-4 adhesion pathway inhibitors used herein may be anti-VLA-4 antibody or VLA-4-binding fragments prepared from the anti-VLA-4 antibody, such as Fab, Fab′, F(ab′)2, and F(v) fragments; heavy chain monomers or dimers; light chain monomers or dimers; and dimers consisting of one heavy chain and one light chain are also contemplated herein. Such antibody fragments may be produced by chemical methods, e.g., by cleaving an intact antibody with a protease, such as pepsin or papain, or via recombinant DNA techniques, e.g., by using host cells transformed with truncated heavy and/or light chain genes. Heavy and light chain monomers may similarly be produced by treating an intact antibody with a reducing agent such as dithiothreitol or β-mercaptoethanol or by using host cells transformed with DNA encoding either the desired heavy chain or light chain or both, or, such as, a monoclonal antibody or an antibody fragment thereof.

Any suitable anti-VLA-4 antibodies or VLA-4-binding fragments capable of blocking the VLA-4-mediated adhesion pathway may be used herein, which include, without limitation, natalizumab and those disclosed in U.S. Pat. No. 6,602,503 and U.S. Patent Application Publication No. US20140161794 A1, which are incorporated herein by reference in their entirety.

In certain embodiments, the VLA-4 adhesion pathway inhibitors used herein may be VLA-4 antagonists that are capable of blocking the VLA-4-mediated adhesion pathway. Exemplary VLA-4 antagonists used herein include, without limitation, VLA-4 antagonists from Tocris Bioscience (e.g., BIO1211, TCS2314, BIO5192, and TR14035).

Inasmuch as VCAM-1 and fibronectin are ligands of VLA-4, the VLA-4 adhesion pathway inhibitors also may include antagonists (including antibodies) of VCAM-1 or fibronectin.

### Pharmaceutical Compositions

Further disclosed herein are pharmaceutical compositions comprising a T cell redirection therapeutic, as disclosed above, and a VLA-4 adhesion pathway inhibitor, as disclosed above, and a pharmaceutically acceptable carrier. Polynucleotides, polypeptides, host cells, and/or engineered immune cells of the invention and compositions comprising them are also useful in the manufacture of a medicament for therapeutic applications mentioned herein. In certain embodiments, the pharmaceutical compositions are separate compositions comprising a T cell redirection therapeutic, as disclosed above, and a VLA-4 adhesion pathway inhibitor, as disclosed above, and a pharmaceutically acceptable carrier. In other embodiments, the pharmaceutical compositions are not separate compositions and the pharmaceutical compositions comprises a T cell redirection therapeutic, as disclosed above, and a VLA-4 adhesion pathway inhibitor, as disclosed above, and a pharmaceutically acceptable carrier.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition according to the invention. According to particular embodiments, in view of the present disclosure, any pharmaceutically acceptable carrier suitable for use in a polynucleotide, polypeptide, host cell, and/or engineered immune cell pharmaceutical composition can be used in the invention.

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include: buffers, diluents, solvents, tonicity regulating agents, preservatives, stabilizers, and chelating agents. One or more pharmaceutically acceptable carrier may be used in formulating the pharmaceutical compositions of the invention.

In one embodiment of the disclosure, the pharmaceutical composition is a liquid formulation. A preferred example of a liquid formulation is an aqueous formulation, i.e., a formulation comprising water. The liquid formulation can comprise a solution, a suspension, an emulsion, a microemulsion, a gel, and the like.

In one embodiment, the pharmaceutical composition can be formulated as an injectable which can be injected, for example, via an injection device (e.g., a syringe or an infusion pump). The injection can be delivered subcutaneously, intramuscularly, intraperitoneally, intravitreally, or intravenously, for example.

In another embodiment, the pharmaceutical composition is a solid formulation, e.g., a freeze-dried or spray-dried composition, which can be used as is, or whereto the physician or the patient adds solvents, and/or diluents prior to use.

### Methods of Use

In another general aspect, the invention relates to a method of treating a cancer in a subject in need thereof, comprising administering to the subject pharmaceutical compositions comprising the T cell redirection therapeutic and the VLA-4 adhesion pathway inhibitor as disclosed herein.

In another general aspect, the invention relates to a method of killing cancer cells comprising subjecting the cancer cells to compositions comprising the T cell redirection therapeutic and the VLA-4 adhesion pathway inhibitor, as disclosed herein.

The subject may have a newly diagnosed cancer or is relapsed or refractory to a prior anti-cancer therapy. The cancer may be a hematological malignancy or a solid tumor.

According to embodiments of the invention, the pharmaceutical compositions comprise a therapeutically effective amount of the T cell redirection therapeutic and the VLA-4 adhesion pathway inhibitor as disclosed herein. As used herein, the term “therapeutically effective amount” refers to an amount of an active ingredient or component that elicits the desired biological or medicinal response in a subject. A therapeutically effective amount can be determined empirically and in a routine manner, in relation to the stated purpose.

As used herein with reference to the T cell redirection therapeutic and the VLA-4 adhesion pathway inhibitor, a therapeutically effective amount means an amount of the T cell redirection therapeutic in combination with the VLA-4 adhesion pathway inhibitor that modulates an immune response in a subject in need thereof. Also, as used herein with reference to the T cell redirection therapeutic, a therapeutically effective amount means an amount of the T cell redirection therapeutic with the VLA-4 adhesion pathway inhibitor that results in treatment of a disease, disorder, or condition; prevents or slows the progression of the disease, disorder, or condition; or reduces or completely alleviates symptoms associated with the disease, disorder, or condition.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

As used herein, the terms “treat,” “treating,” and “treatment” are all intended to refer to an amelioration or reversal of at least one measurable physical parameter related to a cancer, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or more preferably a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

According to particular embodiments, provided are pharmaceutical compositions used in the treatment of a cancer. For cancer therapy, the provided pharmaceutical compositions can be used in combination with another treatment including, but not limited to, a chemotherapy, an anti-CD20 mAb, an anti-TIM-3 mAb, an anti-LAG-3 mAb, an anti-EGFR mAb, an anti-HER-2 mAb, an anti-CD19 mAb, an anti-CD33 mAb, an anti-CD47 mAb, an anti-CD73 mAb, an anti-DLL-3 mAb, an anti-apelin mAb, an anti-TIP-1 mAb, an anti-FOLR1 mAb, an anti-CTLA-4 mAb, an anti-PD-L1 mAb, an anti-PD-1 mAb, other immuno-oncology drugs, an antiangiogenic agent, a radiation therapy, an antibody-drug conjugate (ADC), a targeted therapy, or other anticancer drugs.

According to particular embodiments, the methods of treating cancer in a subject in need thereof comprise administering to the subject T cell redirection therapeutic in combination with a VLA-4 adhesion pathway inhibitor as disclosed herein.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., the T cell redirection therapeutic described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., the VLA-4 adhesion pathway inhibitor) to a subject.

### Kits

In another general aspect, provided herein are kits, unit dosages, and articles of manufacture comprising the T cell redirection therapeutic as disclosed herein, the VLA-4 adhesion pathway inhibitor as disclosed herein, and optionally a pharmaceutical carrier. In certain embodiments, the kit preferably provides instructions for its use.

In another particular aspect, provided herein are kits comprising (1) a T cell redirection therapeutic as disclosed herein, and (2) a VLA-4 adhesion pathway inhibitor as disclosed herein.

In another particular aspect, provided herein are kits comprising pharmaceutical compositions comprising a pharmaceutically acceptable carrier and (1) a T cell redirection therapeutic as disclosed herein, and (2) a VLA-4 adhesion pathway inhibitor as disclosed herein.

### EMBODIMENTS

Embodiment 1 of the invention includes pharmaceutical compositions comprising a T cell redirection therapeutic and a VLA-4 adhesion pathway inhibitor, wherein, the T cell redirection therapeutic comprises a first binding region that immunospecifically binds a T cell surface antigen and a second binding region that immunospecifically binds a tumor associated antigen (TAA).

Embodiment 2 of the invention includes pharmaceutical compositions of embodiment 1, further comprising a pharmaceutically acceptable carrier.

Embodiment 3 of the invention includes pharmaceutical compositions of embodiments 1 or 2, wherein the T cell redirection therapeutic is an antibody or antigen-binding fragment thereof.

Embodiment 4 of the invention includes pharmaceutical compositions of any one of embodiments 1-3, wherein the T cell surface antigen is selected from the group consisting of CD3, CD2, CD4, CD5, CD6, CD8, CD28, CD40L, CD44, CD137, KI2L4, NKG2E, NKG2D, NKG2F, BTNL3, CD186, BTNL8, PD-1, CD195, and NKG2C.

Embodiment 5 of the invention includes pharmaceutical compositions of embodiment 4, wherein the T cell surface antigen is CD3.

Embodiment 6 of the invention includes pharmaceutical compositions of any one of embodiments 1-5, wherein the TAA is selected from the group consisting of BCMA, CD123, GPRC5D, CD33, CD19, PSMA, TMEFF2, CD20, CD22, CD25, CD52, ROR1, HM1.24, CD38, and SLAMF7.

Embodiment 7 includes pharmaceutical compositions of embodiment 6, wherein the T cell redirection therapeutic is a BCMAxCD3 bispecific antibody having a first antigen-binding site that immunospecifically binds BCMA and a second antigen-binding site that immunospecifically binds CD3.

Embodiment 8 includes pharmaceutical compositions of embodiment 7, wherein the BCMAxCD3 bispecific antibody comprises a first heavy chain (HC1), a first light chain (LC1), a second heavy chain (HC2), and a second light chain (LC2), and wherein the HC1 and the LC1 pair to form the first antigen-binding site and the HC2 and the LC2 pair to form the second antigen-binding site.

Embodiment 9 includes pharmaceutical compositions of embodiment 8, wherein the HC1 comprises the amino acid sequence of SEQ ID NO: 1, the LC1 comprises the amino acid sequence of SEQ ID NO: 2, the HC2 comprises the amino acid sequence of SEQ ID NO: 3, and the LC2 comprises the amino acid sequence of SEQ ID NO: 4.

Embodiment 10 includes pharmaceutical compositions of embodiment 8, wherein the HC1 comprises the amino acid sequence of SEQ ID NO: 5, the LC1 comprises the amino acid sequence of SEQ ID NO: 6, the HC2 comprises the amino acid sequence of SEQ ID NO: 3, and the LC2 comprises the amino acid sequence of SEQ ID NO: 4.

Embodiment 11 of the invention includes pharmaceutical compositions of embodiment 6, wherein the T cell redirection therapeutic is a CD123×CD3 bispecific antibody having a first antigen-binding site that immunospecifically binds CD123 and a second antigen-binding site that immunospecifically binds CD3.

Embodiment 12 of the invention includes pharmaceutical compositions of embodiment 11, wherein the CD123×CD3 bispecific antibody comprises a first heavy chain (HC1), a first light chain (LC1), a second heavy chain (HC2), and a second light chain (LC2), and wherein the HC1 and the LC1 pair to form the first antigen-binding site and the HC2 and the LC2 pair to form the second antigen-binding site.

Embodiment 13 of the invention includes pharmaceutical compositions of embodiment 12, wherein the HC1 comprises the amino acid sequence of SEQ ID NO: 7, the LC1 comprises the amino acid sequence of SEQ ID NO: 8, the HC2 comprises the amino acid sequence of SEQ ID NO: 9, and the LC2 comprises the amino acid sequence of SEQ ID NO: 10.

Embodiment 14 of the invention includes pharmaceutical compositions of any one of embodiments 1-13, wherein the VLA-4 adhesion pathway inhibitor is an anti-VLA-4 antibody or antigen-binding fragment thereof.

Embodiment 15 of the invention includes pharmaceutical compositions of embodiment 14, wherein the anti-VLA-4 antibody or antigen-binding fragment thereof is selected from the group consisting of monoclonal antibodies, scFv, Fab, Fab′, F(ab′)2, and F(v) fragments, heavy chain monomers or dimers, light chain monomers or dimers, and dimers consisting of one heavy chain and one light chain.

Embodiment 16 of the invention includes pharmaceutical compositions of any one of embodiments 1-13, wherein the VLA-4 adhesion pathway inhibitor is a VLA-4 antagonist.

Embodiment 17 of the invention includes pharmaceutical compositions of embodiment 16, wherein the VLA-4 adhesion pathway inhibitor is a VLA-4 antagonist selected from the group consisting of BIO1211, TCS2314, BIO5192, and TR14035.

Embodiment 18 of the invention includes methods of killing cancer cells, comprising subjecting cancer cells to therapeutically effective amounts of pharmaceutical compositions of any one of embodiments 1-17 wherein cancer cells undergo some form of cell death.

Embodiment 19 of the invention includes methods of embodiment 18, wherein the T cell redirection therapeutic and the VLA-4 adhesion pathway inhibitor are administered simultaneously or sequentially.

Embodiment 20 of the invention includes methods of embodiment 19, wherein a VLA-4 adhesion pathway inhibitor is administered prior to administration of a T cell redirection therapeutic.

Embodiment 21 of the invention includes methods of embodiment 20, wherein the VLA-4 adhesion pathway inhibitor is administered after administration of the T cell redirection therapeutic.

Embodiment 22 of the invention includes methods of killing cancer cells comprising disrupting cell-cell contact between cancer cells and stromal cells, comprising subjecting cancer cells to therapeutically effective amounts of pharmaceutical compositions of any one of embodiments 1-17 wherein cancer cells undergo some form of cell death.

Embodiment 23 of the invention includes methods of embodiment 22, wherein the T cell redirection therapeutic and the VLA-4 adhesion pathway inhibitor are administered simultaneously or sequentially.

Embodiment 24 of the invention includes methods of embodiment 22, wherein a VLA-4 adhesion pathway inhibitor is administered prior to administration of a T cell redirection therapeutic.

Embodiment 25 of the invention includes methods of embodiment 22, wherein a VLA-4 adhesion pathway inhibitor is administered after administration of a T cell redirection therapeutic.

Embodiment 26 includes methods of killing cancer cells comprising increasing T cell-dependent cytotoxicity, comprising subjecting cancer cells to therapeutically effective amounts of pharmaceutical compositions of any one of embodiments 1-17 wherein cancer cells undergo some form of cell death.

Embodiment 27 of the invention includes methods of embodiment 26, wherein a T cell redirection therapeutic and a VLA-4 adhesion pathway inhibitor are administered simultaneously or sequentially.

Embodiment 28 of the invention includes methods of embodiment 26, wherein a VLA-4 adhesion pathway inhibitor is administered prior to administration of a T cell redirection therapeutic.

Embodiment 29 of the invention includes methods of embodiment 26, wherein a VLA-4 adhesion pathway inhibitor is administered after administration of a T cell redirection therapeutic.

Embodiment 30 of the invention includes methods of killing cancer cells comprising disrupting cell-cell contact between cancer cells and stromal cells and increasing T cell-dependent cytotoxicity, comprising subjecting cancer cells to therapeutically effective amounts of pharmaceutical compositions of any one of embodiments 1-17 wherein cancer cells undergo some form of cell death.

Embodiment 31 of the invention includes methods of embodiment 30, wherein a T cell redirection therapeutic and a VLA-4 adhesion pathway inhibitor are administered simultaneously or sequentially.

Embodiment 32 of the invention includes methods of embodiment 30, wherein a VLA-4 adhesion pathway inhibitor is administered prior to administration of a T cell redirection therapeutic.

Embodiment 33 of the invention includes methods of embodiment 30, wherein a VLA-4 adhesion pathway inhibitor is administered after administration of a T cell redirection therapeutic.

Embodiment 34 of the invention includes methods of altering immunosuppression in a tumor microenvironment, comprising subjecting a tumor microenvironment to therapeutically effective amounts of pharmaceutical compositions of any one of embodiments 1-17 wherein immunosuppression is lessened in the tumor microenvironment and cancer cells undergo some form of cell death.

Embodiment 35 of the invention includes methods of embodiment 34, wherein a T cell redirection therapeutic and a VLA-4 adhesion pathway inhibitor are administered simultaneously or sequentially.

Embodiment 36 of the invention includes methods of embodiment 34, wherein a VLA-4 adhesion pathway inhibitor is administered prior to administration of a T cell redirection therapeutic.

Embodiment 37 of the invention includes methods of embodiment 34, wherein a VLA-4 adhesion pathway inhibitor is administered after administration of a T cell redirection therapeutic.

Embodiment 38 of the invention includes methods of altering immunosuppression in a tumor microenvironment comprising disrupting cell-cell contact between cancer cells and stromal cells, comprising subjecting a tumor microenvironment to therapeutically effective amounts of pharmaceutical compositions of any one of embodiments 1-17 wherein immunosuppression is lessened in the tumor microenvironment and cancer cells undergo some form of cell death.

Embodiment 39 includes methods of embodiment 38, wherein the T cell redirection therapeutic and the VLA-4 adhesion pathway inhibitor are administered simultaneously or sequentially.

Embodiment 40 of the invention includes methods of embodiment 38, wherein a VLA-4 adhesion pathway inhibitor is administered prior to administration of a T cell redirection therapeutic.

Embodiment 41 of the invention includes methods of embodiment 38, wherein a VLA-4 adhesion pathway inhibitor is administered after administration of a T cell redirection therapeutic.

Embodiment 42 of the invention includes methods of altering immunosuppression in a tumor microenvironment comprising increasing T cell-dependent cytotoxicity, comprising subjecting a tumor microenvironment to therapeutically effective amounts of pharmaceutical compositions of any one of embodiments 1-17 wherein wherein immunosuppression is lessened in the tumor microenvironment and cancer cells undergo some form of cell death.

Embodiment 43 of the invention includes methods of embodiment 42, wherein a T cell redirection therapeutic and a VLA-4 adhesion pathway inhibitor are administered simultaneously or sequentially.

Embodiment 44 of the invention includes methods of embodiment 42, wherein a VLA-4 adhesion pathway inhibitor is administered prior to administration of a T cell redirection therapeutic.

Embodiment 45 of the invention includes methods of embodiment 42, wherein a VLA-4 adhesion pathway inhibitor is administered after administration of a T cell redirection therapeutic.

Embodiment 46 of the invention includes methods of altering immunosuppression in a tumor microenvironment comprising disrupting cell-cell contact between cancer cells and stromal cells and increasing T cell-dependent cytotoxicity, comprising subjecting a tumor microenvironment to therapeutically effective amounts of pharmaceutical compositions of any one of embodiments 1-17 wherein immunosuppression is lessened in the tumor microenvironment and cancer cells undergo some form of cell death.

Embodiment 47 of the invention includes methods of embodiment 46, wherein a T cell redirection therapeutic and a VLA-4 adhesion pathway inhibitor are administered simultaneously or sequentially.

Embodiment 48 of the invention includes methods of embodiment 46, wherein a VLA-4 adhesion pathway inhibitor is administered prior to administration of a T cell redirection therapeutic.

Embodiment 49 of the invention includes methods of embodiment 46, wherein a VLA-4 adhesion pathway inhibitor is administered after administration of a T cell redirection therapeutic.

Embodiment 50 of the invention includes methods of treating cancer in a subject in need thereof, comprising administering to the subject a therapeutically effective amount of the pharmaceutical composition of anyone of claims 1-17.

Embodiment 51 of the invention includes methods of embodiment 50, wherein the subject has a newly diagnosed cancer.

Embodiment 52 of the invention includes methods of embodiment 51, wherein the subject is relapsed or refractory to a prior anti-cancer therapy.

Embodiment 53 of the invention includes methods of any one of embodiments 50-52, wherein the cancer is a hematological malignancy or a solid tumor.

Embodiment 54 of the invention includes methods of embodiment 53, wherein the subject has AML or MM.

Embodiment 55 of the invention includes methods of embodiment 50-54, wherein the T cell redirection therapeutic and the VLA-4 adhesion pathway inhibitor are administered simultaneously or sequentially.

Embodiment 56 of the invention included methods of embodiment 55, wherein a VLA-4 adhesion pathway inhibitor is administered prior to the administration of a T cell redirection therapeutic.

Embodiment 57 of the invention includes methods of embodiment 55, wherein a VLA-4 adhesion pathway inhibitor is administered after administration of a T cell redirection therapeutic.

Embodiment 58 of the invention includes kits comprising pharmaceutical compositions of anyone of claims 1-17.

Embodiment 59 of the invention includes kits comprising pharmaceutical compositions of anyone of claims 1-17 wherein the pharmaceutical compositions are packaged separately.

Embodiment 60 of the invention includes kits comprising pharmaceutical compositions of anyone of claims 1-17 wherein the pharmaceutical compositions are packaged together.

### EXAMPLES

### Materials and Methods

### Antibody Design

Bispecific antibodies were produced targeting human CD123 and CD3 or targeting human BCMA and CD3, in which the anti-CD123 or anti-BCMA antibody and the anti-CD3 antibody were joined together post-purification by generating a controlled fragment antigen binding arm exchange using the Genmab technology (17, 18). This resulted in a monovalent binding, bi-functional DuoBody® antibody which specifically binds to human CD123⁺ AML or human BCMA⁺ MM cells and CD3 T cells (FIGS. 8A and 8B). To minimize antibody-mediated effector functions, mutations were introduced in the Fc domain to reduce interactions with Fcγ receptors. The bispecific antibodies (BCMAxCD3 bispecific and CD123×CD3 bispecific) used in the following experiments comprise a first heavy chain (HC1), a second heavy chain (HC2), a first light chain (LC1), and a second light chain (LC2), in which HC1 and LC1 pair to form a first antigen-binding site that immunospecifically binds CD123 or BCMA, and HC2 and LC2 pair to form a second antigen-binding site that immunospecifically binds CD3. The BCMAxCD3 bispecific used in the following examples comprises HC1 having the amino acid sequence of SEQ ID NO: 1, LC1 having the amino acid sequence of SEQ ID NO: 2, HC2 having the amino acid sequence of SEQ ID NO: 3, and LC2 having the amino acid sequence of SEQ ID NO: 4. The CD123×CD3 bispecific used in the following examples comprises HC1 having the amino acid sequence of SEQ ID NO: 7, LC1 having the amino acid sequence of SEQ ID NO: 8, HC2 having the amino acid sequence of SEQ ID NO: 9, and LC2 having the amino acid sequence of SEQ ID NO: 10.

### In Vitro and Ex Vivo Cytotoxicity Assays

Tumor cell lines were labelled with Carboxyfluorescein succinimidyl ester (CFSE) and co-cultured with thawed purified frozen T cells in the presence or absence of stromal cell lines (HS-5 and HS-27a), primary mesenchymal stromal cells (MSC) and CD105⁺ endothelial cells. 24 hours later, bispecific antibodies were added to the wells and the plates were incubated at 37° C. with 5% CO₂ for 48 hours. The cells were then stained for various markers before analyzing on the flow cytometers. For the trans-well related experiments, the assay was performed in 96 well U bottom plates with or without 0.4 μm transwell inserts (HTS TRANSWL96, Corning). For the IncuCyte® related experiments, red fluorescent OCI-AML5 cells (OCI-AML5-NucLight Red) and green HS-5 (HS-5-NucLight Green) were used.

For the ex vivo assays, HS-5 cells were plated prior to addition of AML peripheral blood mononuclear Cells (PBMCs) or MM bone marrow mononuclear cells (BMMCs). CD123×CD3 or BCMA×CD3 or null×CD3 bispecific antibodies (1 μg/ml) with or without anti-VLA4 antibody (5 μg/ml) were added. 72 hours later, depletion of CD123⁺ blasts or CD138⁺ MM plasma cells was monitored via flow cytometry. Additionally, expansion of CD8

T cells as well as their activation status (upregulation of CD25) were assessed.

### In Vivo MOLM-13 Xenograft Model

Human PBMC (1×107 cells/mouse) were inoculated intravenously (iv) 6-7 days prior to tumor cell implantation. On study day 0 mice were implanted subcutaneously (sc) with 1×106 MOLM-13 cells and two concentrations of HS-5 bone marrow stromal cells, 2×105 and 5×105. Treatments with CD123×CD3 (0.04 mg/kg and 0.008 mg/kg, n=8) or vehicle PBS controls (n=5) were given intravenously (iv) every three days (q3d) for 5 doses. Individual mice were monitored for body weight loss and tumor growth inhibition twice weekly for the duration of the study. In the case of the in vivo study with the VLA-4 blocking antibody, treatments with CD123×CD3 bispecific antibody (0.008 mg/kg, n=8 or 9) or PBS vehicle control (n=5) were given iv and the anti-VLA-4 antibody (5 mg/kg) given intraperitoneally (ip). No animals were excluded from the analysis.

### Statistical Methods

Data were analyzed by GraphPad software Prism version 8 (SAS Institutes, Cary, N.C.). Browne-Forsythe and Welch ANOVA test analysis was applied for FIGS. 1 and 2 while ordinary 2-way ANOVA analysis was applied to FIGS. 3-7.

### Cell Lines

KG1, H929, RPMI-8226, MM.1S, HS-5 and HS-27a cell lines were obtained from the American Tissue Culture Collection (Manassas, Va.). MOLM 13 and OCI-AML5 were obtained from Deutsche Sammlung von Mikroorganismen and Zellkulturen (DSMZ, Germany). Primary mesenchymal stem cells cryopreserved from normal human donors were purchased from Lonza (Basel, Switzerland) and CD105⁺ bone marrow endothelial cells were purchased from All Cells (Alameda, Calif.). IncuCyte® NucLight Green or NucLight Red Lentivirus Reagent (EF1a, Puro) was purchased from Essen Bioscience (Ann Arbor, Mich.) and was used according to manufacturer's instructions to generate HS-5-NucLight Green and OCI-AML5-NucLight Red cells. Puromycin treatment was used to select fluorescent positive cell lines. While the cell lines were not authenticated recently, they tested negative for mycoplasma contamination.

Binding Assay with Bispecific Antibodies

All tumor cells were centrifuged, washed twice with Dulbecco's phosphate-buffered saline (DPBS) and 1×104 cells were added to the center of each well of a 96 well U bottom plate along with fragment crystallizable (Fc) block (human IgG1 fragment) which was added at 2 mg/mL for 10 minutes. Serially diluted bispecific antibodies were added to the appropriate wells. Plates were incubated in the dark at 37° C. with 5% CO₂ for 4 hours. The cells were then washed with DPBS and binding of the bispecific antibody was detected by staining with mouse anti-human IgG4 (Southern Biotech, clone HP6025, catalog #9200-09) and LIVE/DEAD (L/D; Invitrogen, catalog #L34976) for 30 minutes. Finally, cells were washed, resuspended in stain buffer, and analyzed on the FACSCanto II flow cytometer (BD Biosciences). Geometric mean fluorescence intensity (gMFI) was plotted in Prism version 7 (GraphPad). The X axis was log transformed and a 4 parameter non-linear curve fit was applied.

In Vitro Cytotoxicity Assays with Cell Lines

Tumor cell lines (KG1, MOLM 13, OCI-AML5, H929, RPMI-8226 and MM.1S) were counted and washed with DPBS before incubation with CFSE (resuspended in 150 dimethyl sulfoxide and diluted 1:10,000) at 1×107 cells/mL of CFSE for 8 minutes at RT. Staining was quenched with HI FBS. Cells were washed in complete medium before resuspension at 2×10⁵ cells/mL in complete medium containing 1 mg/mL human IgG1 fragment, then incubated for 15 minutes. The purified frozen T cells (obtained from BioIVT (Westport, N.Y.)) were thawed and resuspended at 1×10⁶ cells/mL. The T cells were isolated from whole blood by using Ficoll gradient (to isolate mononuclear cells) and negative selection post incubation at room temperature with an antibody cocktail (CD16, CD19, CD36, CD56 and CD66b) to remove the ‘unwanted’ cells. The stroma cell lines (HS-5 and HS-27a) were harvested, washed, counted and resuspended at 4×10⁵ cells/mL. In the case of primary mesenchymal stromal cells (MSC) and CD105⁺ endothelial cells, frozen aliquots sourced from Lonza and All cells respectively, were thawed and resuspended at 4×10⁵ cells/mL. Finally, 50 μL of purified T cells, 50 μL of stromal cells and 100 μL of labeled tumor cells were combined in each well of a 96 well U bottom plate with 0.5 mg/mL human IgG1 fragment. 24 hours later, the test antibodies were added to the wells. The antibodies were diluted to a final starting concentration of 133 nM in DPBS or complete medium. The antibodies were further diluted 3-fold and added to appropriate wells. All plates were incubated at 37° C. with 5% CO2 for 48 hours post addition of antibodies. The cells were then washed with DPBS and stained for various markers before analyzing on the flow cytometers.

For the proliferation experiments, the in vitro assays were performed as detailed above except that here T cells were labelled with the CFSE dye prior to co-culture, thus allowing assessment of proliferation by monitoring CFSE 96 hours post addition of the bispecific antibodies.

For the transwell related experiments, the assay was performed in 96 well U bottom plates with or without 0.4 μM transwell inserts (HTS TRANSWL96, Corning). The stromal cells were either combined with T and tumor cells or separated from the T and tumor cells by seeding on the transwell insert.

For the IncuCyte® related experiments, red fluorescent OCI-AML5 cells were used (OCI-AML5-NucLight Red) and green HS-5 (HS-5-NucLight Green). Tumor, stroma and T cells were washed and combined in phenol-red-free RPMI/10% HI FBS for these assays. Images of red and green objects (indicating red OCI-AML5 and green HS-5) per well were recorded by the IncuCyte® Zoom every 6 hours over a time course of 120 hours.

For blocking experiments, the following inhibitors and neutralizing antibodies were used: Bcl-2 inhibitor (HA14-1), anti-human CXCR4 (12G5) and anti-human ITGA4/VLA4 (2B4) antibody were purchased from R&D systems (Minneapolis, Minn.).

Ex Vivo Cytotoxicity Assays with Primary AML and NM Patient Samples

30,000 or 600,000 HS-5 cells were plated per well of a 6 well plate overnight. Next morning, media was carefully removed before replacing with 3×10⁶ primary AML or MM PBMCs and BMMC, respectively in αMEM+10% FBS with 0.5 mg/mL human IgG1 fragment. Next, CD123×CD3, BCMA×CD3 or null×CD3 bispecific antibodies (1 μg/ml) with or without anti-VLA4 antibody (5 μg/ml) were added. 72 hours later, depletion of CD123⁺ blasts or CD138⁺ MM plasma cells was monitored via flow cytometry. Additionally, expansion of CD8 T cells as well as their activation status (upregulation of CD25) were assessed.

### Flow Cytometry and Antibody Reagents

Antibodies for FACS included the following anti-human antibodies: CD278/ICOS (DX-29), CD4 (SK3), Granzyme B (GB11) (purchased from BD Biosciences), CD8 (RPA-T8), 41BB/CD137 (4B4-1), CD25 (BC96), Perforin (dG9), Tbet (4b10), PD-1/CD279 (EH12.2H7), TIM3 (F38-2E2), CD33 (WM53), CD38 (HIT2), CD123 (6H6), CD138 (MI15) (purchased from BioLegend), LAG3 (3DS223H) (purchased from eBiosciences) and LIVE/DEAD Near-IR (Life Technologies).

For FACS analysis, the plates were centrifuged at 1,500 rpm for 5 minutes. The cells were then washed with DPBS and stained for T cell activation markers and for cytotoxicity for 30 minutes. Finally, cells were washed and resuspended in stain buffer. For intracellular staining, cells were fixed and permeabilised using the IC Staining kit (eBiosciences) according to manufacturer's instructions with minor modifications (washing four times with permeabilization buffer before incubation with intracellular cytokine antibody).

Data was acquired on a FACSCanto II (BD Biosciences) or LSRFortessa ((BD Biosciences). Tumor cell death was assessed by gating on forward-scatter (FSC) and side-scatter (SSC) to identify cell populations, then CFSE⁺ tumor events, and finally LIVE/DEAD Near-IR to assess tumor cell cytotoxicity. The L/D+ gate was drawn after comparing to the PBS treated and isotype controls. These controls also help account for errors related to non-specific binding of antibodies or spillover effects. T cell activation was assessed by gating on FSC and SSC to identify cell populations, CFSE⁻ events, live cells, then looking for positive staining for several markers. The percentage of either dead tumor cells was graphed using Prism 8 and analyzed with a 4 parameter non-linear regression curve fit. For the T cell activation markers, the geometric mean fluorescent intensities of various markers were analyzed via FlowJo software and were graphed using Prism 8.

### Immunoblotting and Antibody Reagents

Automatic western blots were performed using a Wes automated system (ProteinSimple, California, USA) according to manufacturer's instructions. Samples were mixed with a 5× sample buffer containing SDS, DTT and fluorescent molecular weight standards and heated at 95° C. for 5 min and then, loaded onto a plate prefilled with stacking and separation matrices, along with blocking and wash buffers, antibody solutions and detection reagents. Default settings were used for the analysis. The following anti-human antibodies purchased from Cell Signaling Technology (Danvers, Mass.) were used to detect proteins: Bcl-2 (#2872), Phospho-p38 MAPK (Thr180/Tyr182) (D3F9) XP® Rabbit mAb (#4511), Phospho-Akt (Ser473) (D9E) XP® Rabbit mAb (#4060) and β-Actin (D6A8) Rabbit mAb (#8457).

### Animals

Female NSG (NOD scid gamma or NOD.Cg-Prkdcscid Il2rgtm1Wjl/SzJ) mice (The Jackson Laboratory, Bar Harbor, Me.) were utilized when they were approximately 6-8 weeks of age and weighed 20 g. All animals were allowed to acclimate and recover from any shipping-related stress for a minimum of 5 days prior to experimental use. Reverse osmosis (RO) chlorinated water and irradiated food (Laboratory Autoclavable Rodent Diet 5010, Lab Diet) were provided ad libitum, and the animals were maintained on a 12 hour light and dark cycle. Cages, bedding and water bottles were autoclaved before use and changed weekly. All experiments were carried out in accordance with The Guide for the Care and Use of Laboratory Animals and were approved by the Institutional Animal Care and Use Committee of Janssen R&D, Spring House, Pa.

### Results

BM Stromal Cells Protect AML and MM Cell Lines from CD3 Bispecific Antibodies and T Cell-Mediated Cytotoxicity

The BM niche is characterized by its protective and immune-suppressive microenvironment. BM stromal cells were used to mimic the BM niche as they are a major cellular component of the endosteal and vascular niches that govern fundamental hematopoietic stem cells (HSC) cell fate decisions including self-renewal, survival, differentiation, and proliferation (19, 20). BM stromal cells are also documented to mediate immune-suppression (13, 21) while also activating multiple survival and anti-apoptotic pathways in tumor cells, thus allowing them to become resistant to different types of therapy (22). AML or MM cell lines were co-cultured with T cells and bispecific antibodies in the absence or presence of BM stromal cells. Bispecific antibodies targeting either CD123 or BCMA and CD3 (tool antibodies) were used. Binding, killing and T cell activation data demonstrating efficacy of these antibodies are shown in FIG. 8. Using the CD123×CD3 bispecific antibody, dose-dependent killing of CD123-expressing leukemic cell line KG-1 was observed (FIGS. 1A and 1B). This killing was not observed with bispecific antibodies that express either CD3 or CD123 along with a non-targeting (null) arm (FIGS. 1A and 1B). Similar results were observed when using BCMA expressing MM cell line H929 and another bispecific antibody BCMA×CD3 (FIGS. 1C-1D) where specific killing was mediated by BCMA×CD3 in contrast to the null controls. When stromal cells were added to the co-culture, a statistically significant decrease was observed in the maximum observed cytotoxic response, even at high concentrations of the bispecific antibodies (FIGS. 1A-1D). Furthermore, EC50 values of the bispecific antibody were 3-5 fold higher in the presence of stroma (FIG. 9A). Stromal inhibition of bispecific antibody activity was not merely restricted to fibroblast stromal cell lines (HS-5 and HS-27a) but was also observed with primary mesenchymal stromal cells (MSC) derived from the BM of healthy donors (FIGS. 1A-1D). Inhibition of bispecific antibody activity was dependent on the number of stromal cells present in the co-culture; whereby decreased efficacy was still observed when stromal cells were 10 fold less than cancer cells in the co-cultures (FIG. 9B). Interestingly, no inhibition was observed with the addition of CD105⁺ endothelial cells sorted from BM mononuclear cells of healthy donors (FIGS. 1A-1D). This result demonstrated that not all stromal cells adversely impacted the efficacy of CD3 redirection bispecific antibodies and also accounted for the fact that the mere presence of another cell type in the tumor-T cell co-culture did not contribute to the inhibition effect. Stromal-mediated inhibition of bispecific antibody activity was not unique to T cells from one donor but was observed with T cells of multiple donors (means and medians with different donors plotted in FIGS. 1A, C and 1B, D respectively). These data were not specific to one AML or MM cell line as similar results were observed with other CD123⁺ AML (MOLM-13 and OCI-AML5) and BCMA⁺ MM cell lines (RPMI-8226 and MM.1S) (FIGS. 9C and 9D). These data demonstrate for the first time that stromal cells impact the efficacy and potency of CD3 redirection bispecific antibodies.

### BM Stromal Cells Suppress T Cell Activity and Activate Survival and Anti-Apoptotic Pathways in Cancer Cells

Next, the mechanisms underlying stromal inhibition of bispecific antibody activity was investigated. To this end, the phenotype of T cells was assessed in T cell-tumor co-culture cytotoxicity assays in the absence or presence of stromal cells. Treatment with CD123×CD3 bispecific antibody in the absence of stroma resulted in the upregulation of activation markers including CD25, CD137 and ICOS with concomitant increases in checkpoint markers including PD1, LAG3 and TIM3 in CD8⁺ T cells (FIG. 2A). Additionally, the CD8⁺ T cells exhibited characteristics of cytotoxic T lymphocytes (CTL) by increased production of effector proteins such as perforin and granzyme B and upregulation of T-bet expression (FIG. 2A). However, when stromal cells were present in the co-culture, T cells were less activated with reduced expression of activation, effector and checkpoint markers (FIG. 2A). These results were observed with multiple T cell donors (medians with different donors plotted in FIG. 2A) as well as with MM cell line H929 and the BCMAxCD3 bispecific antibody (FIG. 10A). The results with the decreased expression of checkpoint markers on T cells in the presence of stroma may seem counterintuitive at first given that PD1, TIM3 and LAG3 are recognized as inhibitory proteins that regulate T cell activation response. However, these proteins are induced only upon T cell activation and are absent in naive T cells (23-29). Given these data, it is therefore not surprising that the upregulation of PD1, TIM3 and LAG3 is diminished on T cells in the presence of stromal cells and support the less activated phenotype of the T cells in the presence of the inhibitory stromal compartment. Furthermore, T cell proliferation was reduced in the presence of stroma, post-treatment with both bispecific antibodies (FIG. 10B).

In addition to immune suppression, whether stromal-mediated activation of multiple pro-survival and anti-apoptotic pathways in leukemic and myeloma tumor cells could be an additional mechanism to mediate resistance against therapy was investigated (30). Increased phosphorylation of phosphoinositide 3-kinase (PI3K) and Akt and increased protein expression of Bcl-2 in KG-1 cells that were cultured with HS-5 stromal cells and not in KG-1 or HS-5 cells alone were observed (FIG. 2B). Together these data suggest that AML and MM tumor cells can evade T cell-mediated death by a stromal cell dependent mechanism involving activation of resistance pathways in tumor cells in addition to suppressed activation of T cells.

Next, the relative contribution of T cell immune suppression and upregulation of pro-survival pathways to the phenotype of reduced efficacy of CD3 redirection was investigated. Given that Bcl-2 has been directly implicated in survival and resistance of AML and MM cells from several therapies (30, 31), cytotoxicity assays were performed in the presence of stroma with or without the addition of a Bcl-2 inhibitor HA14-1. While the inhibitor successfully prevented expression of Bcl-2 (FIG. 11), it did not rescue stromal-mediated inhibition of CD3 redirection (FIG. 2C) and T cells remained less activated (FIG. 2D). These data support previously published findings where overexpression of Bcl-2 in target cells had minimal impact on the activity of AMG110 (EpCAM×CD3 BiTE) (32).

### Bone Marrow Stromal Cells Attenuate Efficacy of CD3 Redirection In Vivo

Next, whether stromal cells could protect tumor cells from bispecific antibodies-T cell-mediated cytotoxicity in vivo was investigated. To this end, human PBMCs were intravenously inoculated in female NSG mice and one week later, MOLM-13 with or without HS-5 bone marrow stromal cells were implanted subcutaneously (sc) on the flank of the mice. Mice were then treated with CD123×CD3 (8 μg/kg) starting on day 5 post tumor cell implant twice weekly for a total of 5 treatments. Treatment with CD123×CD3 significantly inhibited sc tumor growth (tumor growth inhibition (TGI Day 25)=78%, p<0.0001) in the MOLM-13 alone group compared to PBS or CD3 x null controls (FIG. 3A). This anti-tumor activity was markedly reduced (TGI Day 25=15%) in the presence of stroma and was statistically significant compared to bispecific antibody treated MOLM-13 alone group (p<0.0001) (FIG. 3A). Furthermore, while equal infiltration of CD8⁺ T cells in tumors with or without stroma was observed (FIG. 3B), there were differences in the activation profiles of the T cells that correlated to the presence of stroma (FIG. 3C). CD8⁺ T cells from bispecific antibody treated MOLM-13+HS-5 groups exhibited impaired upregulation of CD25, PD1 and granzyme B compared to the MOLM-13 controls (FIG. 3C). These results support the in vitro observations and strongly suggest that BM stromal cells reduce the efficacy of otherwise potent CD3 redirection therapeutics by suppressing T cell activation.

### Adhesion to Stroma is Critical to Mediate Immune Suppression and Cancer Cell Survival

Stromal cells can mediate immune-suppression and protect tumor cells from cytotoxicity via secretion of soluble factors including immune suppressive mediators such as IL-10, TGF-β and PGE2 or growth factors such as stem cell factor (SCF), IL-7, IL-15, CXCL-12 among others (21, 33). Additionally, stromal cells can directly interact with tumor cells via adhesion pathways inducing resistance (34) and thereby protect malignant cells from T cell-mediated cytotoxicity in a cell-cell contact dependent manner. Visual examination of the cytotoxicity assays revealed that residual leukemic cells not killed by bispecific antibody-T cell-mediated cytotoxicity clustered closely around stromal cells (FIG. 4A), suggesting that cell-cell contact pathways may play an important role in stromal-mediated protection of cancer cells. To discern between soluble vs cell-cell contact dependent mechanisms, in vitro transwell assays were performed to assess if stromal cells could still inhibit efficacy of bispecific antibody-T cell-mediated lysis even if separated from tumor and T cells. It was observed that cell-cell contact played a dominant role in mediating the stromal protection of AML and MM cell lines from bispecific antibody-T cell redirected cytotoxicity since the stromal cells did not exhibit any inhibitory effect when separated from the tumor cells (FIG. 4B and FIG. 12A). Similar trends were observed with T cells from different donors (2 different T cell donors used in FIG. 4B). Moreover, when stromal cells were placed in a trans-well insert, they were unable to suppress T cell activation and expression of perforin, granzyme B and T-bet (FIG. 4C). These data demonstrate the strong dependence on cell-cell interactions for stromal-mediated T cell suppression and protection from T cell-dependent cytotoxicity.

### Blocking VLA4 In Vitro and In Vivo Rescues Stromal-Mediated Inhibition of CD3 Redirection

The adhesion pathways were investigated to determine which one was critical for stromal inhibition of bispecific antibody efficacy. CXCR4 and VLA-4 were focused on because of their documented roles in mediating AML/MM-stroma interactions in the BM (34). Using blocking antibodies against either VLA-4 or CXCR4 (purchased from R&D Systems), it was observed that unlike CXCR4 inhibition which failed to rescue bispecific antibody-mediated cytotoxicity responses in the presence of stroma, VLA-4 inhibition reversed (50-60%) stromal-mediated protection of KG-1 and MOLM-13 from CD123×CD3 bispecific antibody-T cell cytotoxicity (FIG. 5A and FIG. 12B). This effect was more pronounced with H929 and BCMA×CD3 bispecific antibody where VLA-4 inhibition restored cytotoxicity responses (80-100%) even in the presence of stroma (FIG. 5B). Increased cytotoxic responses with VLA-4 inhibition correlated with restored expression of T cell activation markers such as granzyme B and CD25 which were still repressed under the CXCR4 blockade (FIGS. 5C and 5D). This increase in T cell activation markers with VLA-4 inhibition was also statistically significant compared to the untreated counterparts (co-cultures containing either HS-5 or primary MSC stromal cells). VLA-4 inhibition also attenuated the phosphorylation of Akt and PI3K pathways (FIG. 13).

Previous in vivo results had shown that the efficacy of CD123×CD3 was attenuated in treating MOLM-13 tumors with HS-5 bone marrow stromal cells. To determine if the anti-tumor effect can be restored, an anti-VLA-4 neutralizing antibody was combined with CD123×CD3 for the treatment of MOLM-13-bearing mice. Similar to the prior observations, CD123×CD3 (8 μg/kg) promoted a TGI Day 24 of 52.3% (p≤0.0001) compared to PBS treated controls while the same dose of bispecific antibody had minimal effects against MOLM-13 tumors co-injected with HS-5 cells (TGI Day 23=7.6%) (FIG. 6A). However, the concomitant addition of anti-VLA-4 antibody with CD123×CD3 resulted in increased TGI Day 23 of MOLM-13 tumors with HS-5 cells of 48.4%, (p=0.0001) (FIG. 6A). Increased TGI of MOLM-13 tumors with stroma receiving VLA-4 blockade and bispecific antibody treatment was accompanied by improved CD8⁺ T cell activation and effector responses including expression of perforin, CD25 and PD1 (FIG. 6B). The increased TGI and augmented CD8⁺ T cell response was limited to those tumor+HS-5 bearing mice receiving the combination of VLA-4 blockade and bispecific antibody treatment and was absent when the mice were dosed with either agent by themselves. These results strongly suggest that concomitant blockade of VLA-4 along with CD3 redirection agents can overcome the suppressive effects mediated by stromal cells and can mediate superior anti-tumor responses.

### Blocking VLA-4 in Ex Vivo Primary Patient Cultures Restores Efficacy of CD3 Redirection Despite the Presence of Stroma

The findings were then verified with primary frozen/thawed AML and MM samples. Given that primary tumor cells can be a challenge to maintain in culture without exogenous supplementation of cytokines or stromal support, we performed ex vivo cultures of AML/MM samples with varying numbers of stromal cells (representative gating strategy shown in FIGS. 14 and 15). Strikingly, it was observed that there was appreciable and selective killing of CD123⁺ CD33⁺ AML blasts as well as BCMA⁺ CD138⁺ MM tumor cells in CD123×CD3 and BCMA×CD3 bispecific antibody treated cultures that had low stroma: tumor ratio (0.01× HS-5) and not when the cultures were treated with null controls (FIGS. 7A and 7C). The CD123×CD3 killing effect was minimal in cultures with high stroma: tumor ratio (0.2× HS-5, FIGS. 7A and 7C). Additionally, effective cytotoxic responses of clearing primary tumor cells were followed by expansion or activation of CD8⁺ T cells that were restricted to bispecific antibody treated cultures that had less stroma content (FIGS. 7B and 7D). Lastly, when neutralized VLA-4 in combination with CD123×CD3 or BCMA×CD3 was used, superior killing of tumor cells and restoration of efficacy of bispecific antibody despite the higher stromal content in the cultures was observed (FIGS. 7A-D). Blocking VLA-4 along with the bispecific antibody treatment also restored expansion/activation of CD8⁺ T cells in the cultures that had a higher stromal content (FIGS. 7B and 7C). These results were observed with 3 different patients (AML patients in FIG. 7A-B and MM patients in FIG. 7C-D) and strengthen the previous in vitro and in vivo findings. VLA-4 inhibition by itself resulted in the increased depletion of CD123⁺ blasts in the cultures with high stroma: tumor ratio in one of the AML patient samples but this effect was not broadly observed (data not shown). These data indeed demonstrate that combining VLA-4 blockade with CD3 redirection bispecific antibody therapeutics can overcome suppressive effects of stromal cells and provide rationale for exploring such combinations in the clinic.

### Discussion

The complexity of the BM niche has truly been appreciated in the recent years with significant advancements in understanding the molecular and cellular factors that contribute towards maintenance and regulation of hematopoietic stem cells. In the context of hematological malignancies, the same factors can be exploited by cancer stem cells for protection from and resistance to several anti-cancer therapies, thus contributing to minimal residual disease.

The results herein show for the first time how otherwise effective T cell therapeutics can be thwarted by components of the BM microenvironment. Specifically, it was observed that in the presence of BM stromal cells, AML and MM cancer cells were protected from cytotoxicity mediated by T cell and bispecific antibodies. Reduced killing of cancer cells correlated with blunted T cell activation and effector responses. Blocking cell-cell interactions specifically those mediated by the VLA-4 pathway reversed T cell immune suppression leading to increased killing of AML and MM cancer cells. The results thus reaffirm that the BM microenvironment is a formidable factor that needs to be considered even in the context of otherwise potent and effective immune therapies such as CD3 redirection. The results also provide rationale and evidence for combining agents that interfere with adhesion with CD3 redirection therapeutics for better and more complete elimination of MRD.

While it is demonstrated that blocking VLA-4 reverses stromal inhibition of efficacy of bispecific T cell-mediated cytotoxicity and immunosuppression, the mechanisms underlying this regulation remain to be delineated. VLA-4 is expressed on T cells and can provide costimulatory signals resulting in activation of T lymphocytes in addition to mediating adhesion and transendothelial migration of leukocytes (35-38). Clinical studies in multiple sclerosis patients with natalizumab, a humanized monoclonal IgG4 VLA-4 blocking antibody approved for MS, have shown that the drug not only increases the number of CD4⁺ and CD8⁺ T cells in the peripheral blood (39) but also stimulates CD4⁺ and CD8⁺ T cell production of more IL-2, TNF-a, IFN-γ and IL-17 (40-43). While the results were more modest, similar results were observed in vitro where natalizumab induced a mild upregulation of IL-2, IFN-γ and IL-17 expression in activated primary human CD4⁺ T cells propagated ex vivo from healthy donors, suggesting that natalizumab directly acts on T cells (42). The above study was focused on CD4⁺ T cells; so whether the same is observed for CD8⁺ T cells in vitro remains to be investigated. Another mechanism to explain the effect of VLA-4 blockade could be that blocking interaction between tumor and stroma cells disrupts clustering of tumor cells around the stroma, thus allowing the T cells to access the tumor cells, leading to better efficacy of CD3 redirection. Lastly, VLA-4 inhibition has been shown to directly act on AML and MM cells, rendering them more susceptible to chemotherapy and targeted therapies by preventing the expression and upregulation of key pro-survival pathways in the tumor cells themselves (34) or altering tumor cell production of anti-inflammatory cytokines.

While this study was focused on the BM microenvironment, it is possible that a similar phenomenon occurs in solid tumors. Solid tumors contain a complex dense network of extracellular matrix molecules as well as a variety of stromal cell types that may be immunosuppressive.

The results point towards the importance of targeting the BM microenvironment in conjunction with CD3 redirection therapies. Additionally, the results demonstrate that VLA-4 could potentially be used as a biomarker to predict responses toward CD3 redirection and perhaps used to guide patient selection for these immune therapies.

### REFERENCES

All publications cited herein are each hereby incorporated by reference in their entirety.
1. Paietta E. Minimal residual disease in acute myeloid leukemia: coming of age. Hematology American Society of Hematology Education Program. 2012; 2012:35-42.
2. Harousseau J L, Attal M, Avet-Loiseau H. The role of complete response in multiple myeloma. Blood. 2009; 114(15):3139-46.
3. El Rassi F, Arellano M. Update on optimal management of acute myeloid leukemia. Clinical Medicine Insights Oncology. 2013; 7:181-97.
4. Martinez-Lopez J et al. Long-term prognostic significance of response in multiple myeloma after stem cell transplantation. Blood. 2011; 118(3):529-34.
5. Malard F, Harousseau J L, Mohty M. Multiple myeloma treatment at relapse after autologous stem cell transplantation: A practical analysis. Cancer treatment reviews. 2017; 52:41-7.
6. Blatter S, Rottenberg S. Minimal residual disease in cancer therapy—Small things make all the difference. Drug resistance updates: reviews and commentaries in antimicrobial and anticancer chemotherapy. 2015; 21-22:1-10.
7. Basak G W, Srivastava A S, Malhotra R, Carrier E. Multiple myeloma bone marrow niche. Current pharmaceutical biotechnology. 2009; 10(3):345-6.
8. Sanchez-Aguilera A, Mendez-Ferrer S. The hematopoietic stem-cell niche in health and leukemia. Cellular and molecular life sciences: CMLS. 2016.
9. Lane S W, Scadden D T, Gilliland D G. The leukemic stem cell niche: current concepts and therapeutic opportunities. Blood. 2009; 114(6):1150-7.
10. Mercier F E, Ragu C, Scadden D T. The bone marrow at the crossroads of blood and immunity. Nature reviews Immunology. 2011; 12(1):49-60.
11. Noborio-Hatano K et al. Bortezomib overcomes cell-adhesion-mediated drug resistance through downregulation of VLA-4 expression in multiple myeloma. Oncogene. 2009; 28(2):231-42.
12. Green A S et al. Pim kinases modulate resistance to FLT3 tyrosine kinase inhibitors in FLT3-ITD acute myeloid leukemia. Science advances. 2015; 1(8):e1500221.
13. Krause D S, Scadden D T. A hostel for the hostile: the bone marrow niche in hematologic neoplasms. Haematologica. 2015; 100(11):1376-87.
14. Pallasch C P et al. Sensitizing protective tumor microenvironments to antibody-mediated therapy. Cell. 2014; 156(3):590-602.
15. Zhukovsky E A, Morse R J, Maus M V. Bispecific antibodies and CARs: generalized immunotherapeutics harnessing T cell redirection. Current opinion in immunology. 2016; 40:24-35.
16. Topp M S et al. Phase II trial of the anti-CD19 bispecific T cell-engager blinatumomab shows hematologic and molecular remissions in patients with relapsed or refractory B-precursor acute lymphoblastic leukemia. Journal of clinical oncology: official journal of the American Society of Clinical Oncology. 2014; 32(36):4134-40.
17. Labrijn A F et al. Efficient generation of stable bispecific IgG1 by controlled Fab-arm exchange. Proc Natl Acad Sci USA. 2013; 110(13):5145-50.
18. Labrijn A F et al. Controlled Fab-arm exchange for the generation of stable bispecific IgG1. Nat Protoc. 2014; 9(10):2450-63.
19. Rashidi A, Uy G L. Targeting the microenvironment in acute myeloid leukemia. Current hematologic malignancy reports. 2015; 10(2):126-31.
20. Shiozawa Y, Havens A M, Pienta K J, Taichman R S. The bone marrow niche: habitat to hematopoietic and mesenchymal stem cells, and unwitting host to molecular parasites. Leukemia. 2008; 22(5):941-50.
21. Tripodo C et al. The bone marrow stroma in hematological neoplasms—a guilty bystander. Nature reviews Clinical oncology. 2011; 8(8):456-66.
22. Meads M B, Hazlehurst L A, Dalton W S. The bone marrow microenvironment as a tumor sanctuary and contributor to drug resistance. Clinical cancer research: an official journal of the American Association for Cancer Research. 2008; 14(9):2519-26.
23. Chikuma S et al. PD-1-mediated suppression of IL-2 production induces CD8+ T cell anergy in vivo. J Immunol. 2009; 182(11):6682-9.
24. Inozume T et al. Selection of CD8+PD-1+ lymphocytes in fresh human melanomas enriches for tumor-reactive T cells. J Immunother. 2010; 33(9):956-64.
25. Gros A et al. PD-1 identifies the patient-specific CD8(+) tumor-reactive repertoire infiltrating human tumors. J Clin Invest. 2014; 124(5):2246-59.
26. Simon S et al. PD-1 expression conditions T cell avidity within an antigen-specific repertoire. Oncoimmunology. 2016; 5(1):e1104448.
27. Gros A et al. Prospective identification of neoantigen-specific lymphocytes in the peripheral blood of melanoma patients. Nat Med. 2016; 22(4):433-8.
28. Huard B, Gaulard P, Faure F, Hercend T, Triebel F. Cellular expression and tissue distribution of the human LAG-3-encoded protein, an MHC class II ligand. Immunogenetics. 1994; 39(3):213-7.
29. Hastings W D et al. TIM-3 is expressed on activated human CD4+ T cells and regulates Th1 and Th17 cytokines. Eur J Immunol. 2009; 39(9):2492-501.
30. Matsunaga T et al. Interaction between leukemic-cell VLA-4 and stromal fibronectin is a decisive factor for minimal residual disease of acute myelogenous leukemia. Nat Med. 2003; 9(9):1158-65.
31. Maji S et al. Bcl-2 Antiapoptotic Family Proteins and Chemoresistance in Cancer. Adv Cancer Res. 2018; 137:37-75.
32. Deisting W, Raum T, Kufer P, Baeuerle P A, Munz M. Impact of Diverse Immune Evasion Mechanisms of Cancer Cells on T Cells Engaged by EpCAM/CD3-Bispecific Antibody Construct AMG 110. PloS one. 2015; 10(10):e0141669.
33. Raffaghello L, Vacca A, Pistoia V, Ribatti D. Cancer associated fibroblasts in hematological malignancies. Oncotarget. 2015; 6(5):2589-603.
34. Rashidi A, DiPersio J F. Targeting the leukemia-stroma interaction in acute myeloid leukemia: rationale and latest evidence. Therapeutic advances in hematology. 2016; 7(1):40-51.
35. Sato T, Tachibana K, Nojima Y, D'Avirro N, Morimoto C. Role of the VLA-4 molecule in T cell costimulation. Identification of the tyrosine phosphorylation pattern induced by the ligation of VLA-4. Journal of immunology. 1995; 155(6):2938-47.
36. Davis L S, Oppenheimer-Marks N, Bednarczyk J L, McIntyre B W, Lipsky P E. Fibronectin promotes proliferation of naive and memory T cells by signaling through both the VLA-4 and VLA-5 integrin molecules. Journal of immunology. 1990; 145(3):785-93.
37. Shimizu Y, van Seventer G A, Horgan K J, Shaw S. Costimulation of proliferative responses of resting CD4+ T cells by the interaction of VLA-4 and VLA-5 with fibronectin or VLA-6 with laminin. Journal of immunology. 1990; 145(1):59-67.
38. Mittelbrunn M et al. VLA-4 integrin concentrates at the peripheral supramolecular activation complex of the immune synapse and drives T helper 1 responses. Proceedings of the National Academy of Sciences of the United States of America. 2004; 101(30):11058-63.
39. Marousi S, Karkanis I, Kalamatas T, Travasarou M, Paterakis G, Karageorgiou CE. Immune cells after prolonged Natalizumab therapy: implications for effectiveness and safety. Acta neurologica Scandinavica. 2013; 128(1):e1-5.
40. Bornsen L et al. Effect of natalizumab on circulating CD4+ T-cells in multiple sclerosis. PloS one. 2012; 7(11):e47578.
41. Kivisakk P et al. Natalizumab treatment is associated with peripheral sequestration of proinflammatory T cells. Neurology. 2009; 72(22):1922-30.
42. Benkert T F et al. Natalizumab exerts direct signaling capacity and supports a pro-inflammatory phenotype in some patients with multiple sclerosis. PloS one. 2012; 7(12):e52208.
43. Kimura K et al. Disrupted balance of T cells under natalizumab treatment in multiple sclerosis. Neurology(R) neuroimmunology & neuroinflammation. 2016; 3(2):e210.
44. Yu S et al. Recent advances of bispecific antibodies in solid tumors. Journal of hematology & oncology. 2017; 10(1):155.
45. Ishiguro T et al. An anti-glypican 3/CD3 bispecific T cell-redirecting antibody for treatment of solid tumors. Science translational medicine. 2017; 9(410).
46. Feig C et al. Targeting CXCL12 from FAP-expressing carcinoma-associated fibroblasts synergizes with anti-PD-L1 immunotherapy in pancreatic cancer. Proceedings of the National Academy of Sciences of the United States of America. 2013; 110(50):20212-7.

It is claimed:

1. A pharmaceutical composition comprising a T cell redirection therapeutic and a VLA-4 adhesion pathway inhibitor, wherein, the T cell redirection therapeutic comprises a first binding region that immunospecifically binds a T cell surface antigen and a second binding region that immunospecifically binds a tumor associated antigen (TAA).

2. The pharmaceutical composition of claim 1, further comprising a pharmaceutically acceptable carrier.

3. The pharmaceutical composition of claim 1, wherein the T cell redirection therapeutic is an antibody or antigen-binding fragment thereof.

4. The pharmaceutical composition of claim 1, wherein the T cell surface antigen is selected from the group consisting of CD3, CD2, CD4, CD5, CD6, CD8, CD28, CD40L, CD44, CD137, KI2L4, NKG2E, NKG2D, NKG2F, BTNL3, CD186, BTNL8, PD-1, CD195, and NKG2C.

5. The pharmaceutical composition of claim 4, wherein the T cell surface antigen is CD3.

6. The pharmaceutical composition of claim 1, wherein the TAA is selected from the group consisting of BCMA, CD123, GPRC5D, CD33, CD19, PSMA, TMEFF2, CD20, CD22, CD25, CD52, ROR1, HM1.24, CD38, and SLAMF7.

7. The pharmaceutical composition of claim 6, wherein the T cell redirection therapeutic is a BCMAxCD3 bispecific antibody having a first antigen-binding site that immunospecifically binds BCMA and a second antigen-binding site that immunospecifically binds CD3.

8. The pharmaceutical composition of claim 7, wherein the BCMAxCD3 bispecific antibody comprises a first heavy chain (HC1), a first light chain (LC1), a second heavy chain (HC2), and a second light chain (LC2), and wherein the HC1 and the LC1 pair to form the first antigen-binding site and the HC2 and the LC2 pair to form the second antigen-binding site.

9. The pharmaceutical composition of claim 8, wherein the HC1 comprises the amino acid sequence of SEQ ID NO: 1, the LC1 comprises the amino acid sequence of SEQ ID NO: 2, the HC2 comprises the amino acid sequence of SEQ ID NO: 3, and the LC2 comprises the amino acid sequence of SEQ ID NO: 4.

10. The pharmaceutical composition of claim 8, wherein the HC1 comprises the amino acid sequence of SEQ ID NO: 5, the LC1 comprises the amino acid sequence of SEQ ID NO: 6, the HC2 comprises the amino acid sequence of SEQ ID NO: 3, and the LC2 comprises the amino acid sequence of SEQ ID NO: 4.

11. The pharmaceutical composition of claim 6, wherein the T cell redirection therapeutic is a CD123×CD3 bispecific antibody having a first antigen-binding site that immunospecifically binds CD123 and a second antigen-binding site that immunospecifically binds CD3.

12. The pharmaceutical composition of claim 11, wherein the CD123×CD3 bispecific antibody comprises a first heavy chain (HC1), a first light chain (LC1), a second heavy chain (HC2), and a second light chain (LC2), and wherein the HC1 and the LC1 pair to form the first antigen-binding site and the HC2 and the LC2 pair to form the second antigen-binding site.

13. The pharmaceutical composition of claim 12, wherein the HC1 comprises the amino acid sequence of SEQ ID NO: 7, the LC1 comprises the amino acid sequence of SEQ ID NO: 8, the HC2 comprises the amino acid sequence of SEQ ID NO: 9, and the LC2 comprises the amino acid sequence of SEQ ID NO: 10.

14. The pharmaceutical composition of claim 1, wherein the VLA-4 adhesion pathway inhibitor is an anti-VLA-4 antibody or antigen-binding fragment thereof.

15. The pharmaceutical composition of claim 14, wherein the anti-VLA-4 antibody or antigen-binding fragment thereof is selected from the group consisting of monoclonal antibodies, scFv, Fab, Fab′, F(ab′)2, and F(v) fragments, heavy chain monomers or dimers, light chain monomers or dimers, and dimers consisting of one heavy chain and one light chain.

16. The pharmaceutical composition of claim 1, wherein the VLA-4 adhesion pathway inhibitor is a VLA-4 antagonist.

17. The pharmaceutical composition of claim 16, wherein the VLA-4 antagonist is selected from the group consisting of BIO1211, TCS2314, B105192, and TR14035.

18. A method of killing cancer cells comprising disrupting cell-cell contact between cancer cells and stromal cells, comprising subjecting cancer cells to a therapeutically effective amount of the pharmaceutical composition of claim 1.

19. The method of claim 18, wherein the cancer is a hematological malignancy or a solid tumor.

20. The method of claim 18, wherein the T cell redirection therapeutic and the VLA-4 adhesion pathway inhibitor are administered simultaneously or sequentially.

21. The method of claim 20, wherein the VLA-4 adhesion pathway inhibitor is administered prior to the T cell redirection therapeutic.

22. The method of claim 20, wherein the VLA-4 adhesion pathway inhibitor is administered after administration of the T cell redirection therapeutic.

23. A kit comprising the pharmaceutical composition of claim 1.
